(19) United States
(12) Patent Application Publication
(10) Pub. No.: US 2009/0076519 A1
(43) Pub. Date: Mar. 19, 2009

(54) SYSTEM, METHOD AND TOOL FOR ENSURING CORRECT INSERTION OF AN ARTIFICIAL HIP JOINT

(75) Inventor: Bjorn Franc Iversen, Humlebaek (DK)

Correspondence Address: BROOKS KUSHMAN P.C., 1000 TOWN CENTER, TWENTY-SECOND FLOOR, SOUTHFIELD, MI, 48075, US

(73) Assignee: OM SURGICAL (UK) LIMITED, London (GB)

(21) Appl. No.: 11/914,176

(22) PCT Filed: May 11, 2006
(86) PCT No.: PCT/DK06/00255

§ 371 Date: Oct. 31, 2008

(30) Foreign Application Priority Data
May 12, 2005 (DK) PA 2005 00696
Oct. 27, 2005 (DK) PA 2005 01494

(51) Int. Cl. A61B 17/58

(52) U.S. Cl. 606/99; 606/102

(57) ABSTRACT

The present invention relates to the area of orthopaedic surgery, and in particular to a system for ensuring that prosthesis components or parts thereof are inserted correctly upon implantation of artificial hip joints and to methods for ensuring correct insertion of the parts of an artificial hip joint or femoral prosthesis during surgery. This system comprises a tool (30) for controlling the mutual positioning of the main components in a hip prosthesis, a measuring device (40), for measuring the distance between two supports connected to the patient's pelvis and leg, and connection members (46, 47) arranged at said measuring device, where said connection members are adapted to interact with receptors at said supports.

### FIELD OF THE INVENTION

The present invention relates to the area of orthopaedic surgery, and in particular to a system for ensuring that prosthesis components or parts thereof are inserted correctly upon implantation of artificial hip joints and to methods for ensuring correct insertion of the parts of an artificial hip joint or femoral prosthesis during surgery.

### BACKGROUND OF THE INVENTION

An artificial hip joint has two main components; a prosthesis stem and a socket which is often referred to as “the cup”. One end of the prosthesis stem is provided either with a spherical ball head or a prosthesis neck, which may be modular and designed so that the neck may rotate in the stem, on which stem a ball head may be placed. Said ball head is designed for a close, sliding fit in a spherical recess in the cup. Together, the prosthesis stem/neck with the ball head and the cup will act as a ball joint to replace the natural hip joint.

The other end of the prosthesis stem comprises an elongated part designed to be mounted in the hollow internal canal in the patient's femur.

The cup is designed to be attached in the natural joint socket on the patient's pelvis. The hemispherically shaped, recess in the cup is linked with an external (outer) surface designed to be attached to the pelvis, via a side face. The external surface may have various shapes, all according to the method of attachment to the pelvis and other choices made by the supplier. Several of the cups that are in use are shaped as an approximate hemisphere, where the outer hemispherical surface is designed to be cemented to the pelvis. The side face that connects the recess and the exterior surface may be flat or possibly inwardly sloping towards the recess, which is preferably approximately centered in the side face.

The prosthesis stem and the cup may be fixed to the femur and the pelvis respectively by using cement, or through a cement-free force fit. The invention is intended for and may be used with both fixation techniques but is only described in detail for the cemented version. When replacing a worn out hip with a prosthesis, the cavity on the pelvis is milled out to receive the cup, which is then fixed either by means of poly methyl metacrylate cement or force fit.

The head of the femur is replaced. This is done by dividing the neck of the femur and evacuating approximately the cranial ⅓ of the femoral canal to make room for the elongated prosthesis stem that is either cemented or force fitted into the internal canal.

If the ball head is detachable, this is placed on the prosthesis neck before the ball head is placed in the cup, the joint is assembled by lifting the patient's leg up to a natural position and inserting the ball head in the recess in the cup, whereupon the incision is closed.

Ideally such prostheses should provide the patients with mobility that approximates that which is provided by the natural joint. However, as the tension of the soft tissues surrounding the joint, e.g. the joint capsule etc. is weakened after the operation is possible for the patient to place the leg in a position outside its normal range of movement. This may cause the head of the prosthesis to lever out of the cup (dislocation). Moreover, it is important that “natural” movements of the leg do not bring the joint in positions where the neck of the prosthesis rides on the edge of the cup as this may result in dislocation through simple leverage. Dislocation occurs in 1 to 9% of all patients who have undergone total hip replacement. If this happens, the patient must be anaesthetised before the joint may be reduced. In some patients a re-operation is required. The risk of dislocation is considered increased in patients whose prosthesis components are inserted in an incorrect mutual positioning, than in those where the mutual positioning of the components (i.e. the spatial interface between the components) is correct.

The inventor has previously shown that an optimal mutual relationship between the prosthesis neck and the cup under experimental conditions (not published) results in a reduced risk of dislocation because the patient can go through the everyday natural range of motion (ROM) without the parts of the prosthesis ending up in such mutual positioning so as to entail a risk of dislocation.

The inventor has previously shown (not published) that the most adequate ROM is achieved by assembling both prosthesis components in a manner so as to give them a forward angle of about 15 degrees relative to the frontal plane of the body, while the cup forms an angle of 45 degrees with the horizontal plane. In medical terminology, forward angling is termed anteversion, whereas a backward angling is termed retroversion. A cup angle greater than 45 degrees relative to the horizontal plane is termed abduction, whereas an angle less than 45 degrees is called adduction.

The inventor has also previously shown (not published) that even though the optimum is to have each of the components angled 15 degrees forward, the resulting Range of Motion of the prosthetic joint is nearly as good if the sum of the forward angling of the two components is 30 degrees. Thus a prosthesis joint where the cup is angled forwards at 10 degrees and the prosthesis stem is angled forwards at 20 degrees will result in a ROM for the patient that is nearly as adequate as if both components were angled forward at 15 degrees, the sum of the forward angling being 30 degrees is both cases During the fixation of the prosthesis stem, accurate alignment of the prosthesis stem in the femoral canal may be difficult in practice, especially if the stem is to be fastened cement free. Due to the shape of the internal canal in femur, the prosthesis stem has a tendency to orient itself in accordance with the natural shape of the canal in femur resisting to be forced into the specific angle intended by the surgeon.

Several solutions for insertion of the cup respectively the prosthesis stem and to ensure that the individual part is being fixed correctly are known.

A device for alignment and for holding the cup as it is cemented into the pelvis is known from U.S. Pat. No. 5,976,149. The temporarily holding device for the cup is temporarily fixed to the pelvis during the cementation.

From GB 2.197.790 a device for assuring that the cup in an artificial hip joint is fixed with a predetermined anteversion and a predetermined angle to the horizontal plane, is known. The mutual angle between the parts in the prosthesis is not taken care of by using this device.

Instruments for insertion of the cup are described in EP 888.759 A1 and U.S. Pat. No. 5,540,697.

These instruments are handles onto which the cup is fastened during the insertion but they do not have any means for assuring the correct position and direction of the cup.

Thus, these parameters depend on the individual surgeon, his visual assessment and his experience.

Several devices and means for assuring alignment of the prosthesis stem during the insertion into the femur is know from EP 207 873, PCT/DE90/00715 and EP 865 776 A2. As mentioned this fixation is not critical. Additionally, these publications do only describe devices and means for insertion of one of the prosthesis parts, i.e. the prosthesis stem, and does not describe any means to ensure an intended mutual angle between the cup and the prosthesis stem.

The inventor's own WO 01/19296, that is included as reference in the present description, relates to a tool to set the intended mutual angle between the prosthesis stem and the cup during the cementation of the cup in the pelvic cavity. The tool described may be locked relative to the prosthesis stem and has one or more abutment surface(s) designed to rest against a surface of the cup so that the parts are locked relative to each other. Preferably the prosthesis or its corresponding rasp is fixed to the channel in femur firstly, before the leg of the patient and the prosthesis stem and the device are placed in a normal position and is used to position the cup correctly. This device, however, may not be used by itself to assure the mutual positioning between the prosthesis parts when using cups to be mounted without the use of cement. Additionally it may only be used to assure that the parts of the prosthesis is positioned correctly relative to each other, but does not take into consideration the correct insertion relative to the patient.

The bone coverage for the cup is often inferior when the cup is correctly mounted. The surgeon will often in cases like that choose to deviate from the normally desired angle for the cup to get a better bone coverage. In these cases it would be of great advantage if the surgeon could measure the actual angle and thus be able to choose the best compromise between angle and bone coverage.

The present inventor's own W002/080824, that is included as reference in the present description, describes a computer based method and means that ensure a correct mutual positioning of the main parts of the prosthesis in order to reduce the possibility of errors, and thereby also reduce the risk of dislocation with the resulting pain for the patient, and a possible second operation.

The method and tool according to W002/080824 also makes it possible to accurately measure required adjustments of the length of the limb by inserting the prosthesis so that the resulting leg length may be lengthened or shortened, and to adjust offset, i.e. the distance between the longitudinal axis of the femur and the sagittal plane of the body.

The desired adjustment of the offset and/or length of the limb will be determined during a preoperative examination and outpatient examination of the patient and the patient's radiograms.

This assessment may be sufficient, especially for experienced surgeons who carry out a considerable number of this type of operation each year. But it is estimated that 80% of all implantations of artificial hip joints are carried out by surgeons who do less than 20 of these per year. This number is not sufficient to obtain and maintain the skills and routine required to achieve good surgical results. It is therefore desirable to have a method and means that ensure a correct mutual positioning of the main parts of the prosthesis in order to reduce the possibility of errors, and thereby also reduce the risk complications including dislocation with the resulting pain for the patient, and a possible second operation.

The computer based tool according to W002/080824 is, however, relatively expensive and complicated and requires high technical skills besides the surgical skills. This type of tool is therefore normally utilized by the larger hospitals and mainly university hospitals.

Tools for ensuring the correct insertion of an artificial hip joint, so that the prosthesis is correctly positioned relative to the femur and pelvis and that the parts of the artificial joint are correctly placed relative to each other, are of special importance in minimally invasive surgery. In minimally invasive surgery the operation is performed through relatively small incisions. The main advantage with minimal invasive surgery is that the damage done to healthy tissue is reduced and that the convalescence period becomes shorter. Working through small incisions is, however, more demanding than traditional surgery. For hip joint prosthesis surgery the main problem is related to the position of the parts of the prosthesis. There is a necessity for a method and tools to ensure that the artificial hip prosthesis is correctly inserted during the surgery.

Recently a number of high cost electronic navigation systems for achieving correct alignment of hip prostheses has been developed. As these systems are expensive, complicated and demanding to use they are mainly employed in a limited number of university clinics, while smaller hospitals cannot afford this technology or have not the specially trained human resources to man them. Thus, an inexpensive mechanical invention like the present is in demand to solve the prevailing complications involved with misalignment of the prosthetic components and to spread minimally invasive surgery beyond university clinics.

Leg length discrepancy after total hip replacement is a well known and frequent complication. A couple of devices on the market offer only partial control, leaving the technology open to considerable improvement.

Such a device is described in WO 01/30247, which international application discloses a device for measuring leg length, whereby the length of a patients leg remains the same both prior to and following insertion of a prosthesis. The device according to WO 01/30247 comprises a level fixation means, which may ensure that the foot is pointing upwards, but if the leg of the patient is pointing in another direction after the insertion of the prosthesis than before, this will result in a wrong positioning of the patient's leg. Thus, it is impossible to assure that exactly the same three-dimensional direction of the leg is obtained. It is only possible to measure the length of the patient's leg with the device according to WO 01/30247.

Offset (measured as the shortest distance between the center of the femoral head and a line drawn down the center of femoral shaft) has attracted far less attention even if the effects of reduced offset (medialisation of the femoral shaft) and increased offset (lateralisation of the femoral shaft) are serious. It is desired to restore preoperative hip biomechanics and minimise wear of the artificial hip joint prosthesis. However, it is not in all cases possible to exactly restore preoperative hip biomechanics after replacement of the hip joint due to mechanical limitations of the artificial hip joint prosthesis.

While increased offset after Total Hip Replacement increased Range of Motion by reducing femoropelvic impingement, and increased abductor muscle tension through increased abductor muscle lever arm, complications include increased rotational torque on the prosthesis stem, which may lead to prosthesis loosening.

The offset of a prosthesis joint is difficult to control as a number of prosthesis parameters may influence the result, e.g. stem CCD angle other than the typical 135 deg; medial or lateral shift of the neck union due to valgus or varus orientation of the shaft—or—medialisation or lateralisation of the new joint socket mounted in the pelvis. Also increasing modular neck length, often done to ascertain proper muscle tension over the new joint will increase vertical offset at the same time increasing leg length.

Devices currently claiming to address these problems overlook several aspect of crucial importance for reliable measurements.

WO2004/084740 of same applicant discloses a device addressing these issues to a certain degree. WO2004/084740, which herein is incorporated by reference, discloses a system for ensuring correct insertion and spatial orientation of a prosthesis cup and/or a prosthesis neck of an artificial hip joint. The system comprises a tool for controlling the mutual positioning of the main components in a femoral prosthesis; a measuring device for measuring the distance between two supports connected to the patient's pelvis and femur; and a positioning tool designed to be releasably connected to a handle part connected to the tool according to item a) and to the two supports connected to the patients pelvis and femur. A measuring device and a positioning tool included in the system is also described in addition to a method for surgery by means of the disclosed tool.

However, there is a need for further improvements for advantageous function in all clinical situations, but using the described device has revealed several areas that must be amended for reliable function.

Hence, the measurement tool disclosed in WO2004/084740, and shown in FIG. 5 to 8, is not advantageous in all situations. Therefore, there is a desire to provide a more advantageous tool of this kind. It is desired that this tool provides more flexibility with regard to different reference points made available by the surgeon, as will be described below. Furthermore, the tool should enable a reliable way of compensating for different orientations of the prosthesis stem in the femur.

### SUMMARY OF THE INVENTION

A system is provided for ensuring correct insertion and spatial orientation of a prosthesis cup and/or a prosthesis stem of an artificial hip joint, the system comprising:
a) a tool for controlling the mutual positioning of the main components in a hip prosthesis;
b) a measuring device for measuring the distance between two supports connected to the patient's pelvis and leg, comprising; an elongated main body; a first arm and a second arm, wherein said first and second arm is connected to said elongated main body and at least one of said first and second arm is displaceable along said main body along a first plane, and at least one of said first and second arm is repositionable with regard to said main body along a second plane substantially orthogonal to said first plane, wherein the relation between said first and second plane anteriorly or posteriorly may be shifted along said second plane of the measuring device; connection members arranged at one of the ends of said first and second arm, where said connection members are adapted to interact with receptors at said supports.

A system according to above is provided, further comprising
c) a detachable positioning tool designed to be connected to a handle part connected to the tool according to item a) or to extensions of the prosthesis components, and to the two supports connected to the patients' pelvis and leg.

A measuring device is provided for measuring the distance between two supports for use during surgical procedures, where said supports are connected to bones in the patient's body, wherein the measuring device comprises an elongated main body; a first arm and a second arm, wherein said first and second arm is connected to said elongated main body and at least one of said first and second arm is displaceable along said main body along a first plane, and at least one of said first and second arm is repositionable with regard to said main body along a second plane substantially orthogonal to said first plane, wherein the relation between said first and second plane anteriorly or posteriorly may be shifted along said second plane of the measuring device; connection members arranged at one of the ends of said first and second arm, where said connection members are adapted to interact with receptors at the supports.

Said first and/or second arm being rotatable in said first plane.

Said second arm being positioned on an adjustable member (110).

Said first arm is displaceably connected to the main body in a direction substantially perpendicular to a longitudinal axis of said main body.

According to another preferred embodiment, said second arm is displaceably connected to the adjustable member in a direction substantially perpendicular to the longitudinal axis of said main body.

Furthermore, according to an advantageous improvement, the first and/or second arm is releasably lockable and displaceable/pivotable/rotatable around an axle perpendicular to said longitudinal axis of said main body.

Hence, a more flexible measurement device is provided. The improved device is shown in FIGS. 1A-8A and 9-26 respectively, together with an illustration of its use.

A more detailed use and method is described below.

When using the measuring device, the connection members are brought in contact with the receptors at both supports. The adjustable member and the length of said first or said second arm is then adjusted so that the connection members and the corresponding receptor rests are in full contact with each other. In the illustrated embodiment where the receptors are grooves, the position of the adjustable member and the adjustable arm are adjusted until the connection members rest in the grooves and is in contact with the bottom of the groove at most or all of the length of the connection members. It is preferred that the adjustable member comprises means to lock the adjustable member in a preferred position along the main body. By locking the adjustable member relative to the main body, unintentional movement of the adjustable member relative to the main body after performing the measurement is avoided.

Preferably, the adjustable member is adapted to receive and interact with a locking member to lock the adjustable member to the main body.

Preferably, the measuring device comprises means to lock said first arm and/or second arm in preferred positions and rotational orientations. This is done to avoid unintentional movement of the adjustable arm, i.e. the first arm or the second arm, after performing the measurements.

According to a preferred embodiment, substantially parallel bores are made in the main body and the adjustable member. Said bores provides firm fixation for drill sheaths in using the measuring device as a drill guide and for correct positioning of screws or nails into the patients' femur and pelvis and for fixation of the supports including the receptors. Alternatively, a bent curvature, preferably a hemicircle shape, is provided in the main body of the measurement device in order to provide a drill support surface having easy access.

The improved measurement device provides a more flexible measurement system and provides good alignment of the parts involved in relation to each other in order to provide as much comfort for the surgeon as well as the patient. The measurement device provides both length and orientation measures as a result of its unique advantageous design.

Furthermore, the measuring device provides for reliable compensation of variations of the hip prosthesis stem in the femur.

### DESCRIPTION OF FIGURES

In the following, the invention will be described further with reference to the attached figures, in which:

FIG. 1 shows a tool connected to an anteversion head;

FIG. 2 shows the same tool as FIG. 1, wherein main parts are partly disconnected;

FIG. 3 is a section view of a arm member of the tool seen along the line B-B;

FIG. 4 shows the same tool as FIG. 1, partly disassembled;

FIG. 5 shows an alternative;

FIG. 6 shows an alternative tool;

FIG. 7 shows the tool of FIG. 7 set on the prosthesis stem; and

FIG. 8 shows an alternative version of the present tool, where the tools parts are disassembled.

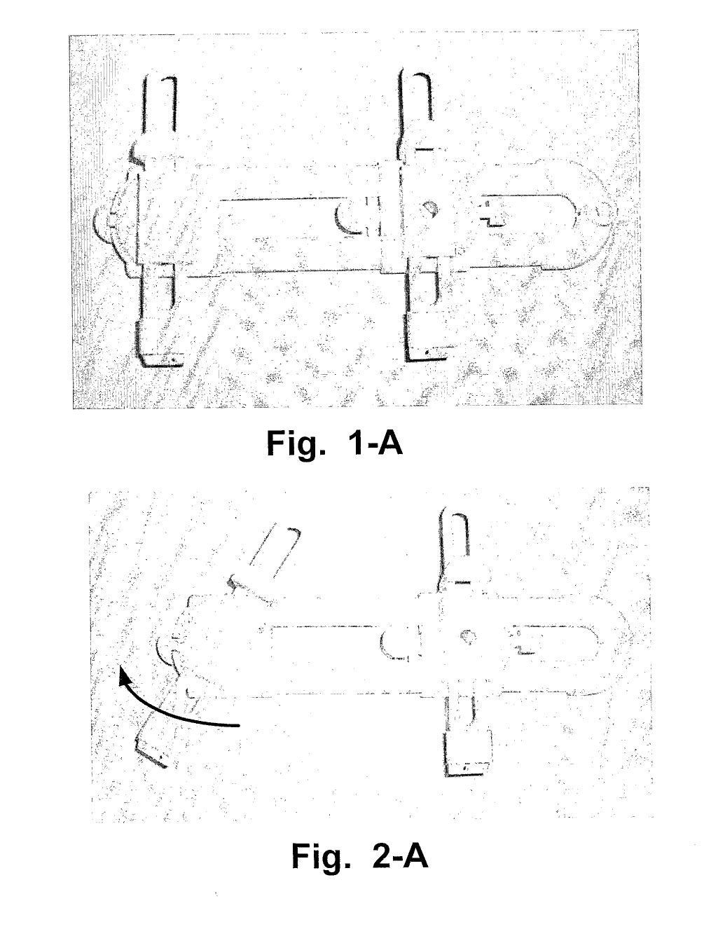

FIGS. 1A-8A and 9 to 26 show another variant of the measurement tool, and

Fig. X1-X20 illustrate a measurement tool according to an embodiment of the present invention.

FIG. 1A shows a measurement tool;

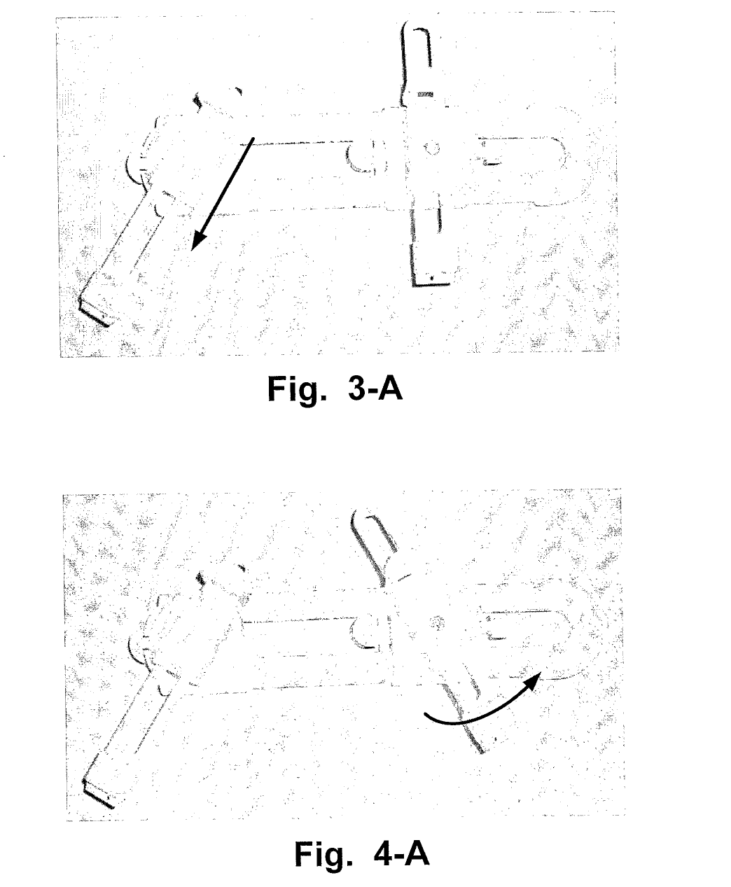

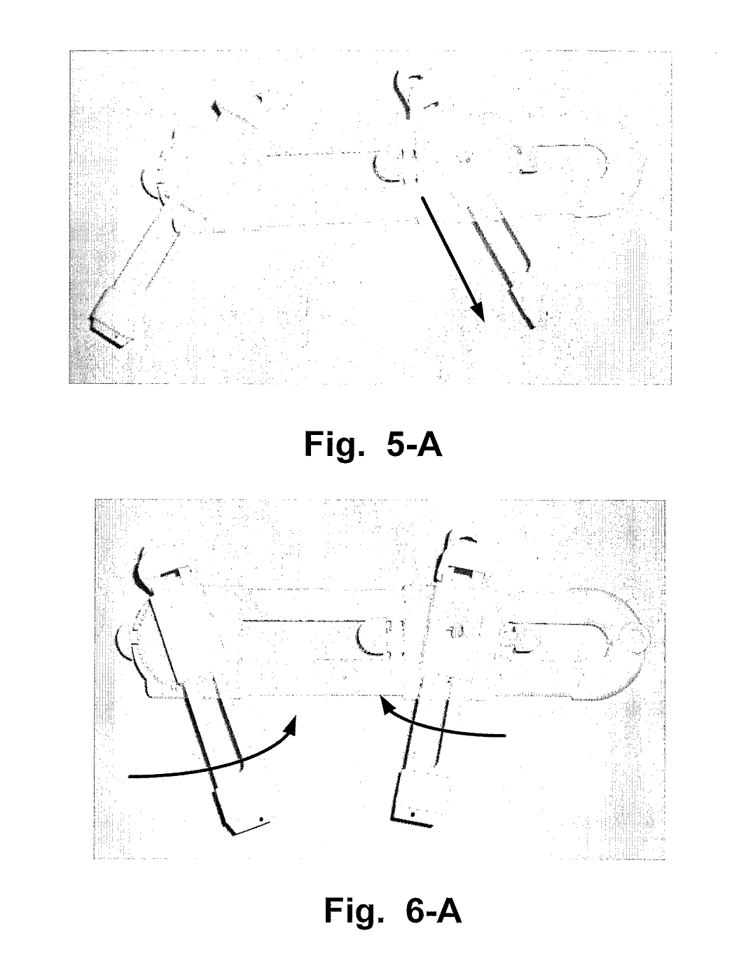

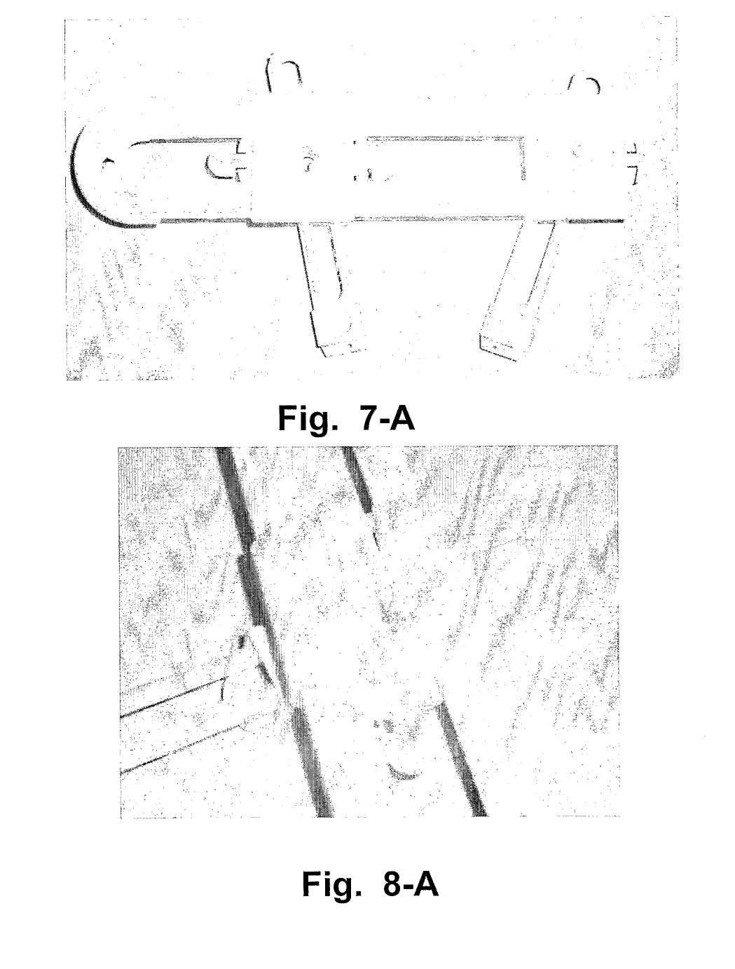

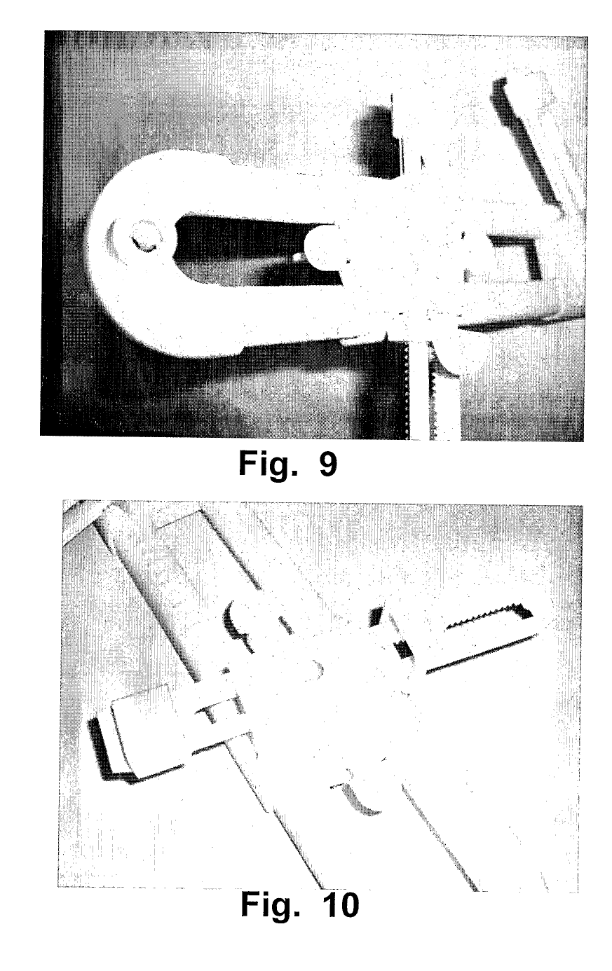

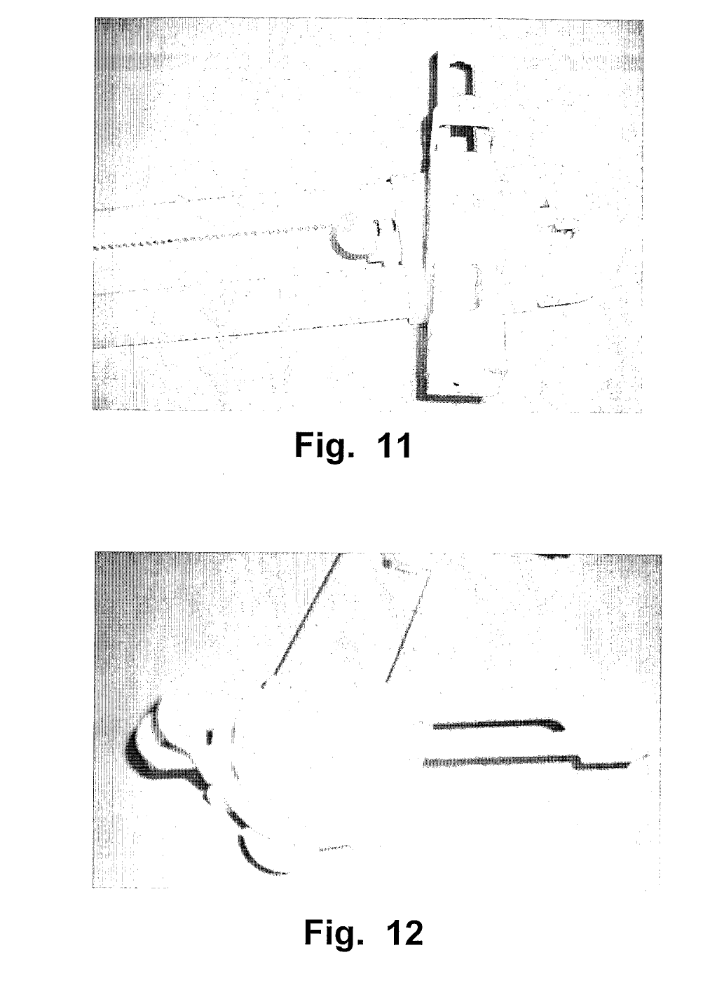

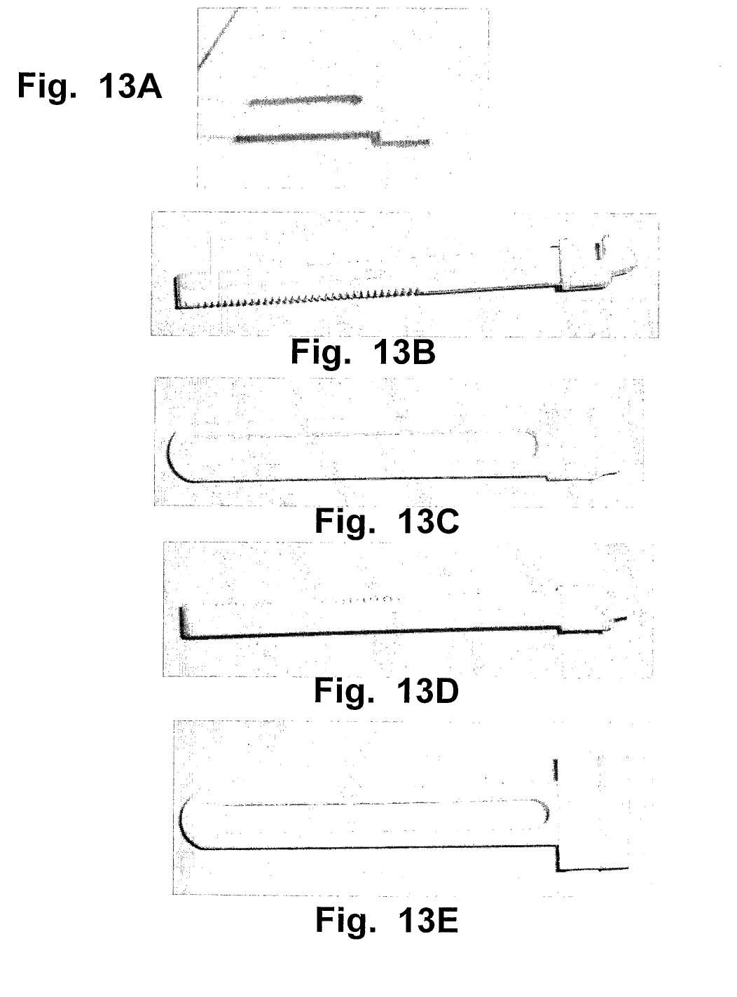

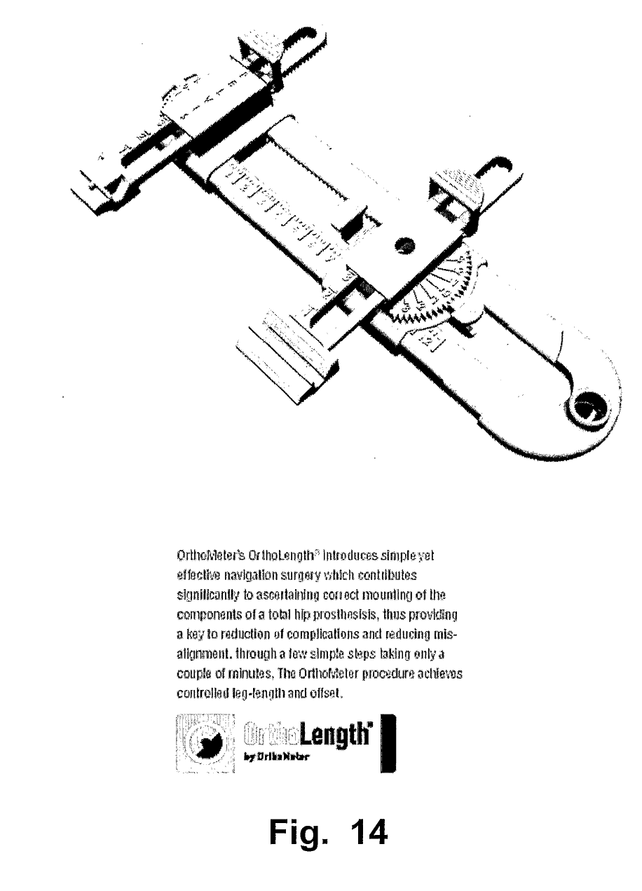

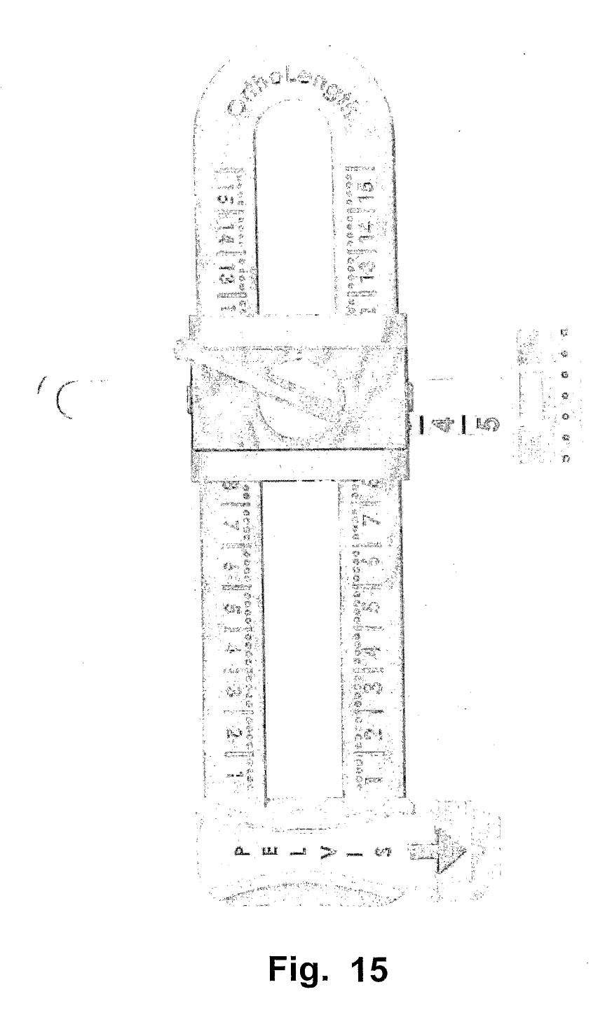

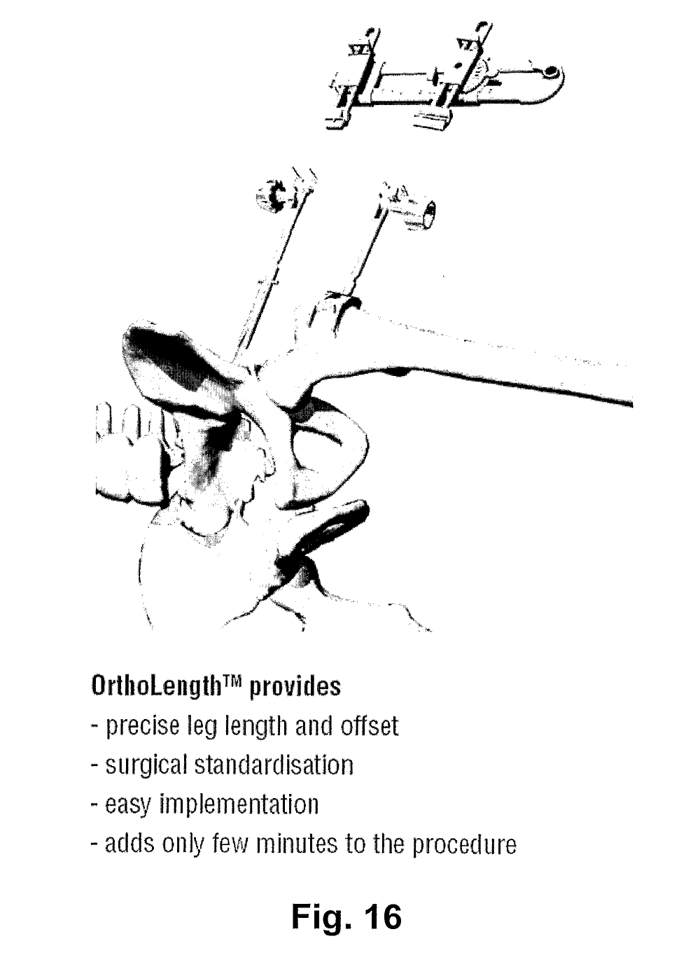

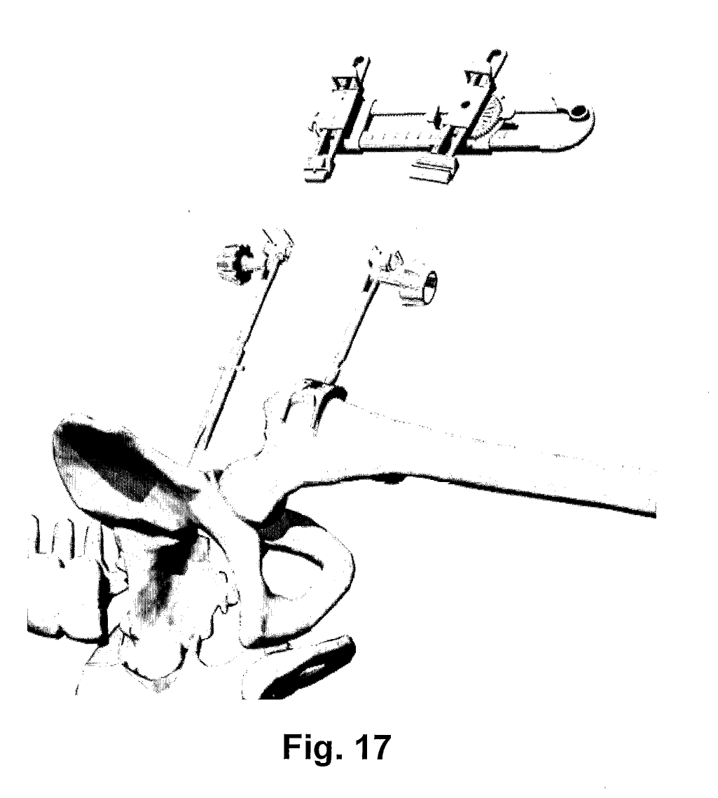

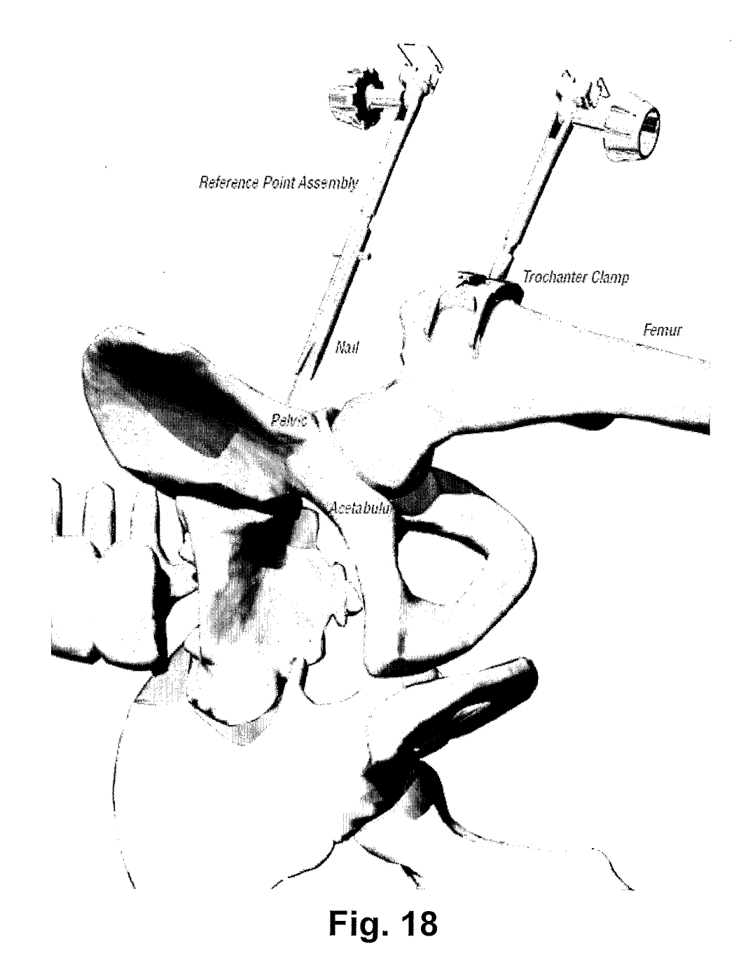

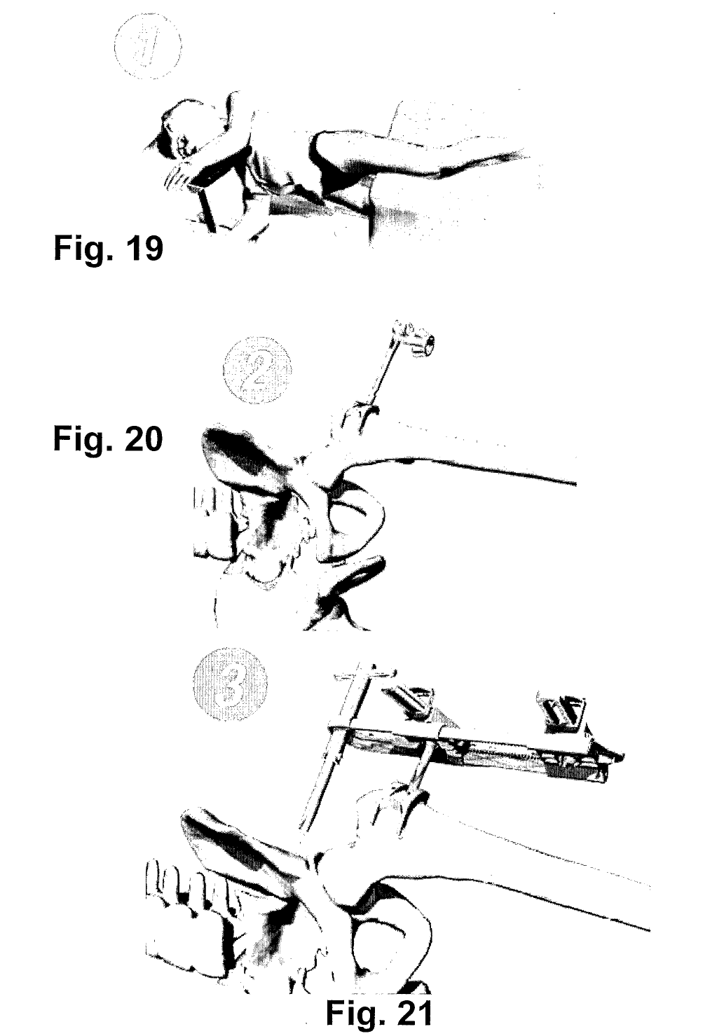

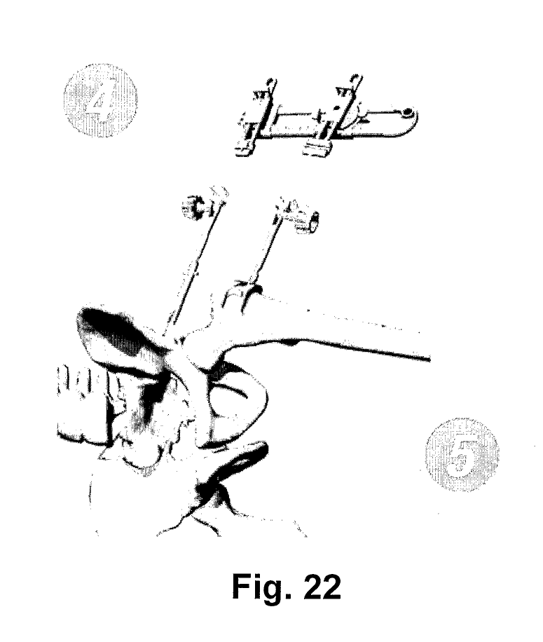

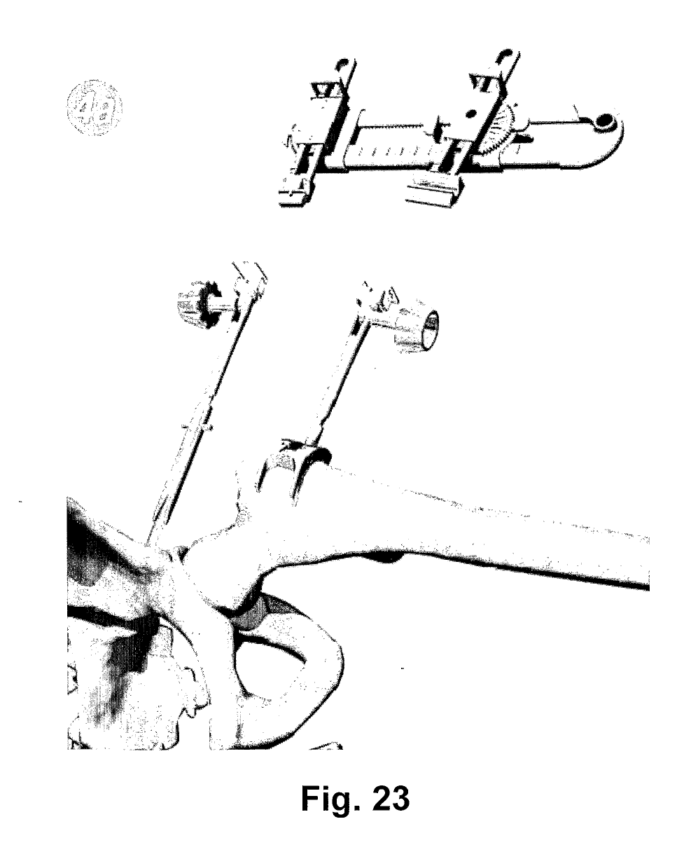

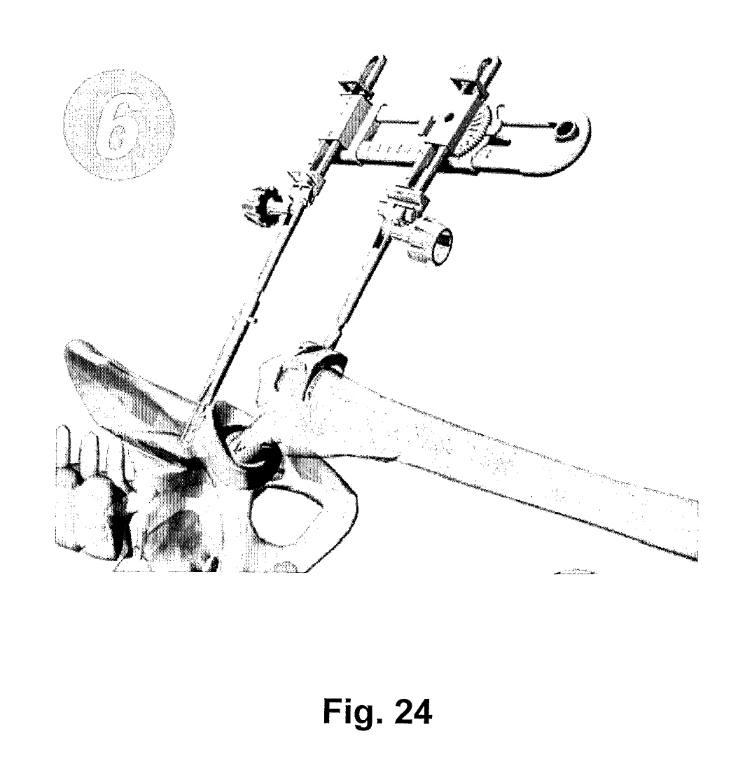

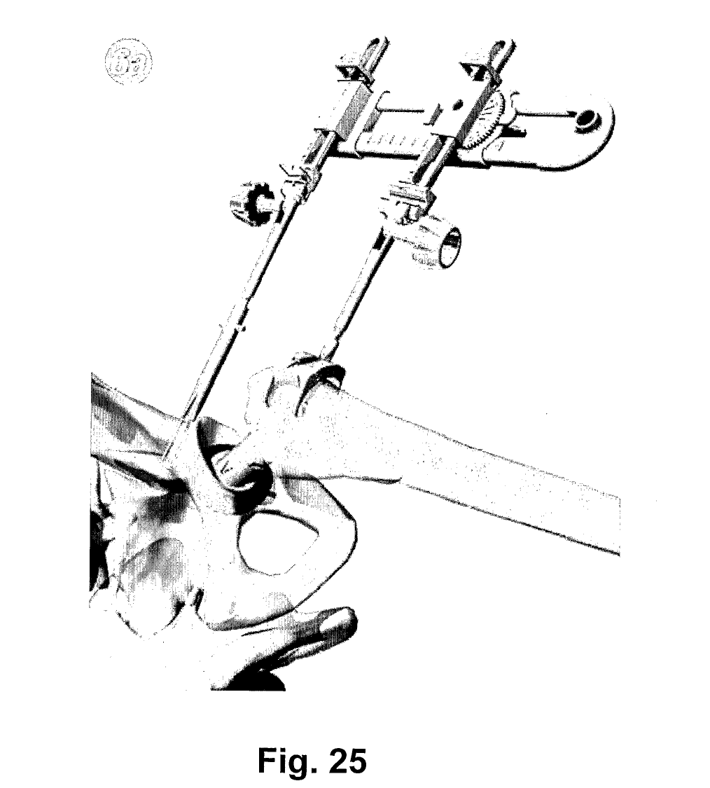

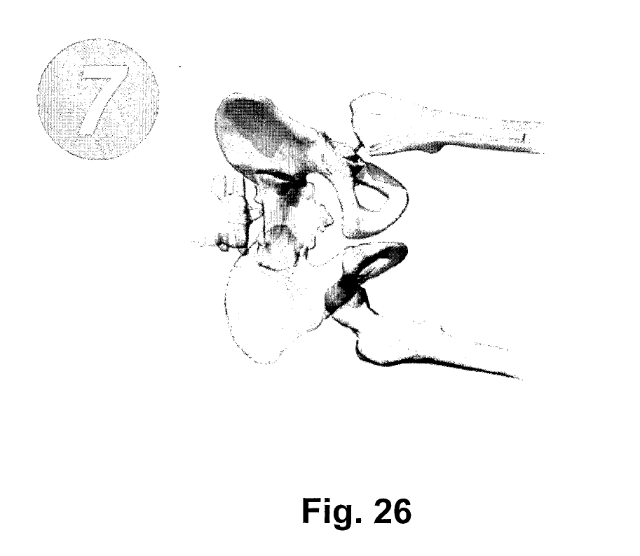

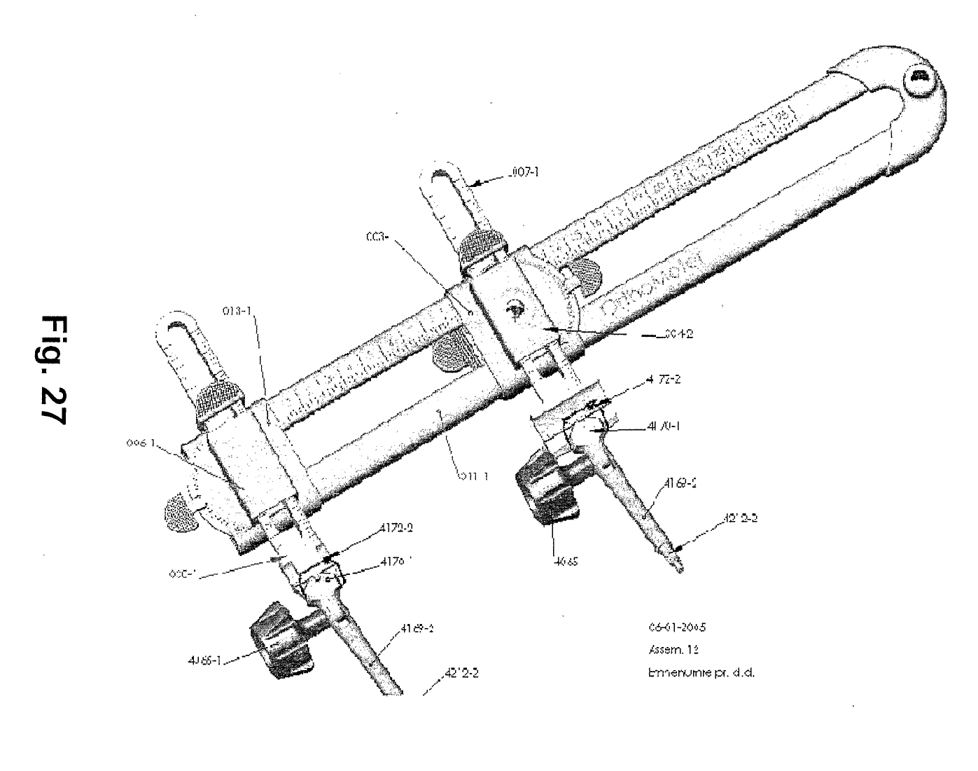

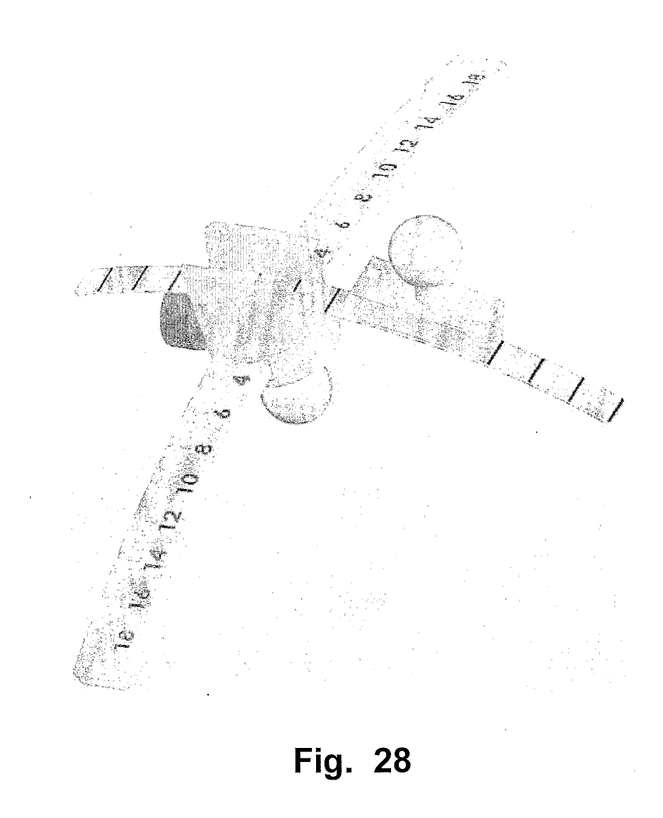

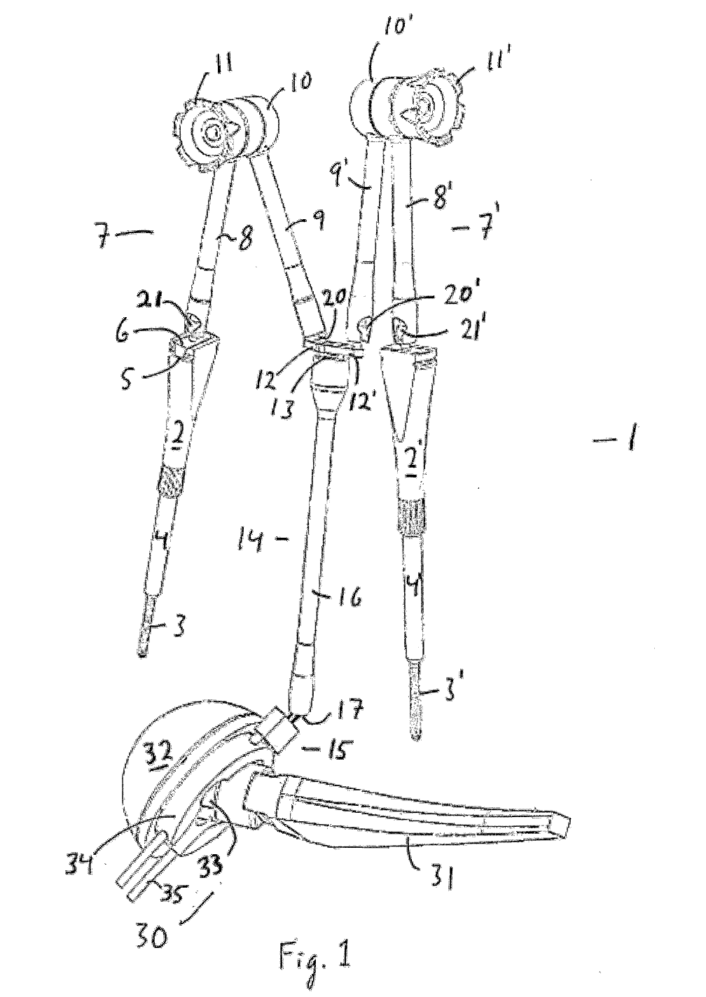

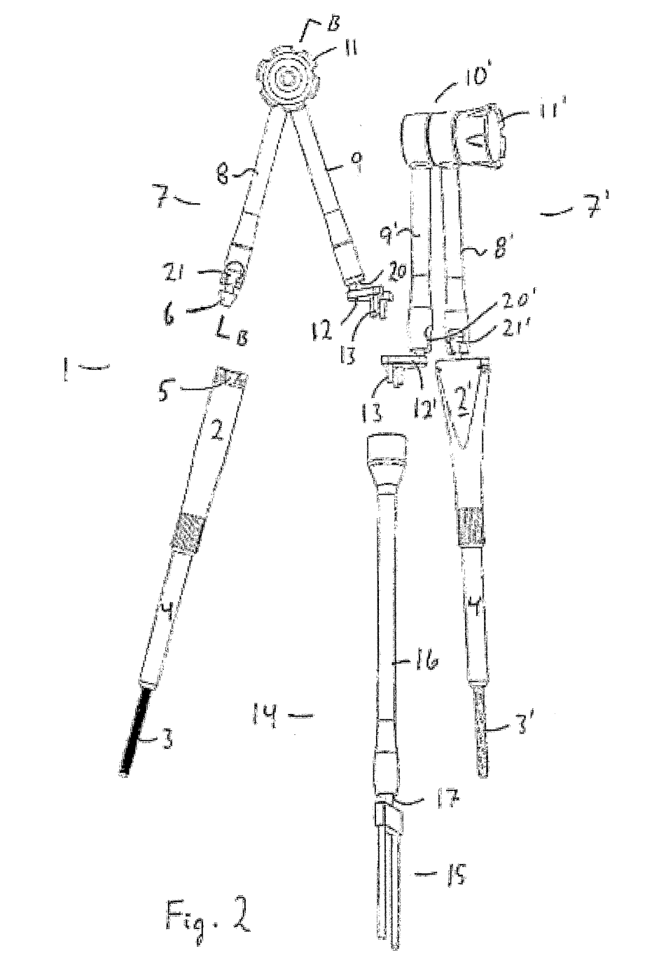

FIG. 2A shows the measurement tool with one arm rotated for better adjustment to a fix point connected to the pelvis;

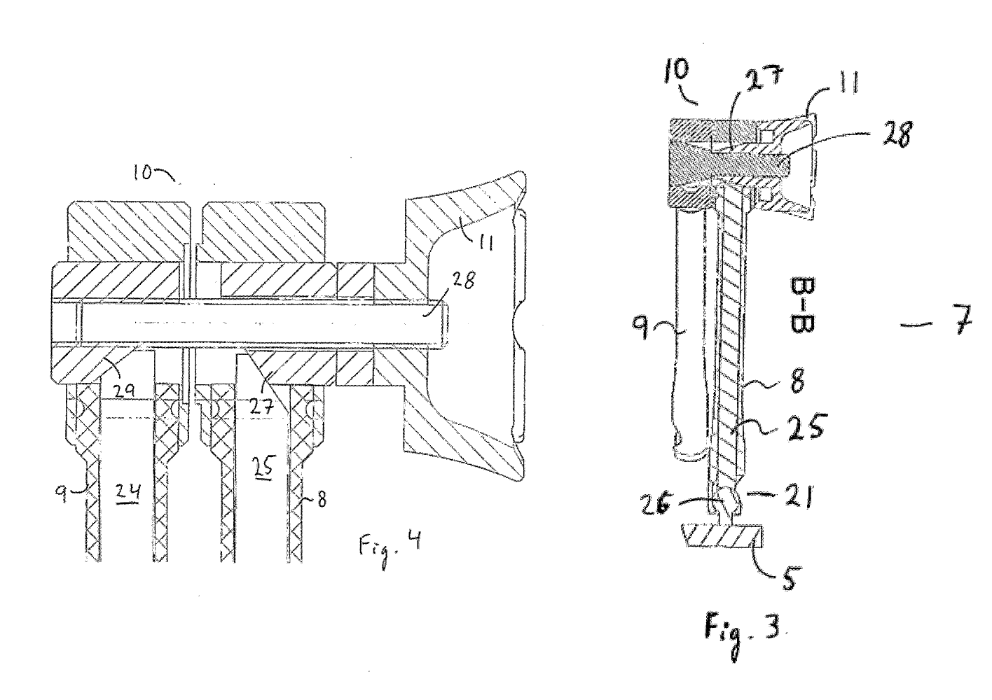

FIG. 3A shows the measurement tool with one arm rotated and extended for better adjustment to a fix point connected to the pelvis;

FIG. 4A shows the measurement tool with one arm rotated and extended as well as the second arm rotated for better adjustment to a fix point connected to the pelvis;

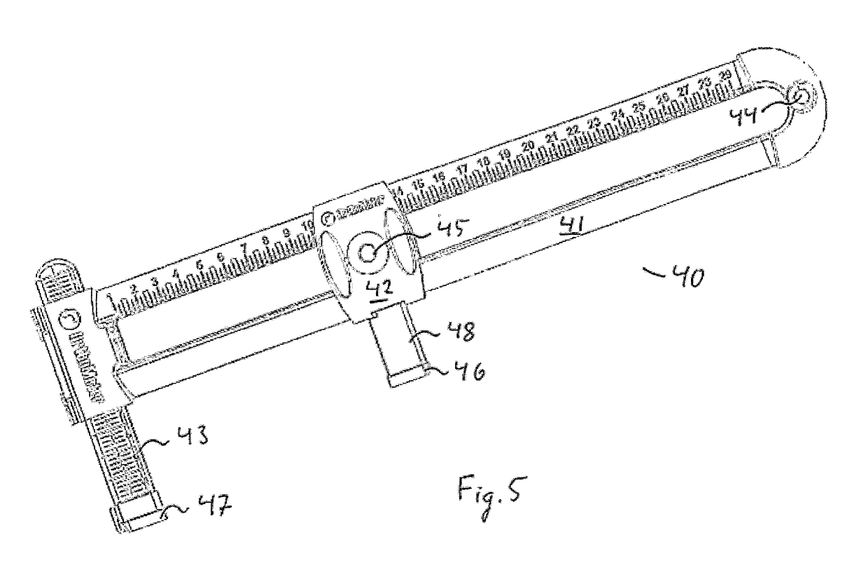

FIG. 5A shows the measurement tool with one arm rotated and extended as well as the second arm rotated and extended for better adjustment to a fix point connected to the pelvis;

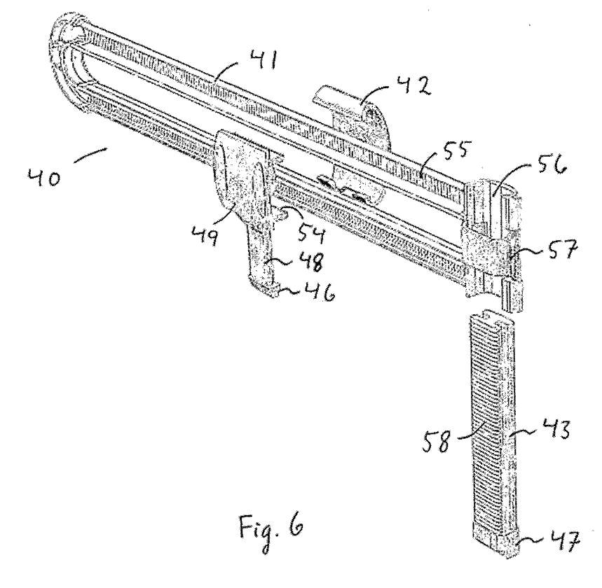

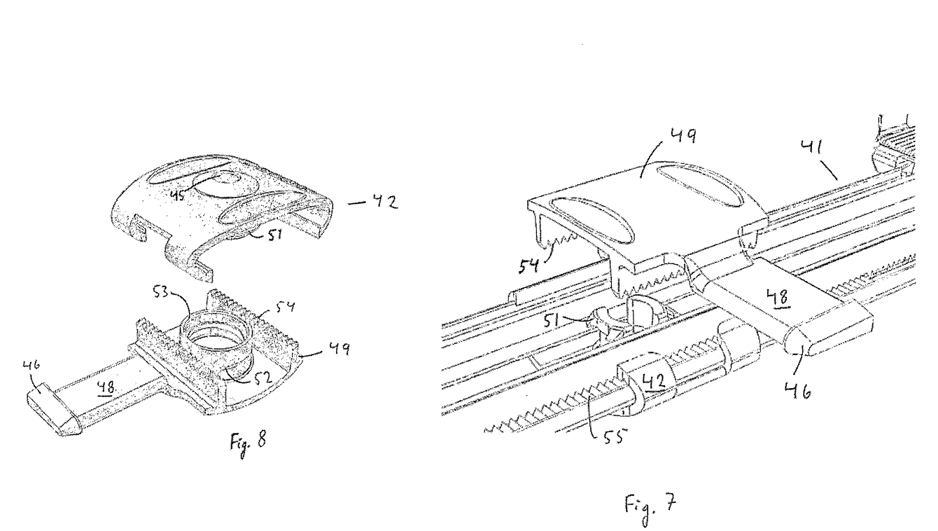

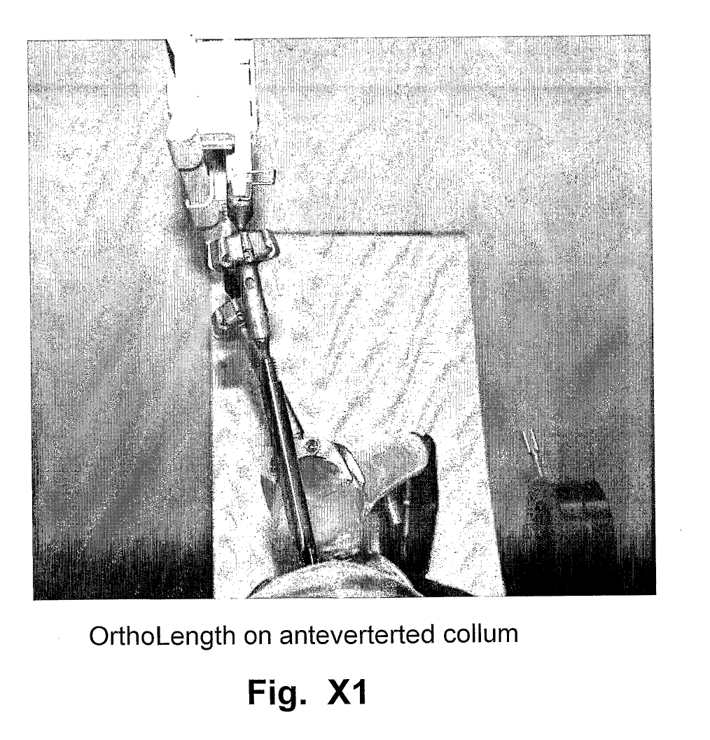

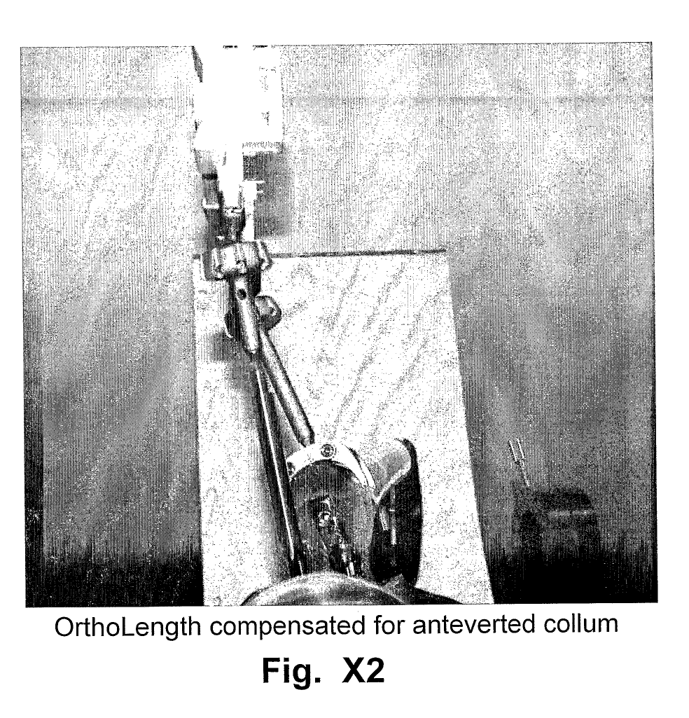

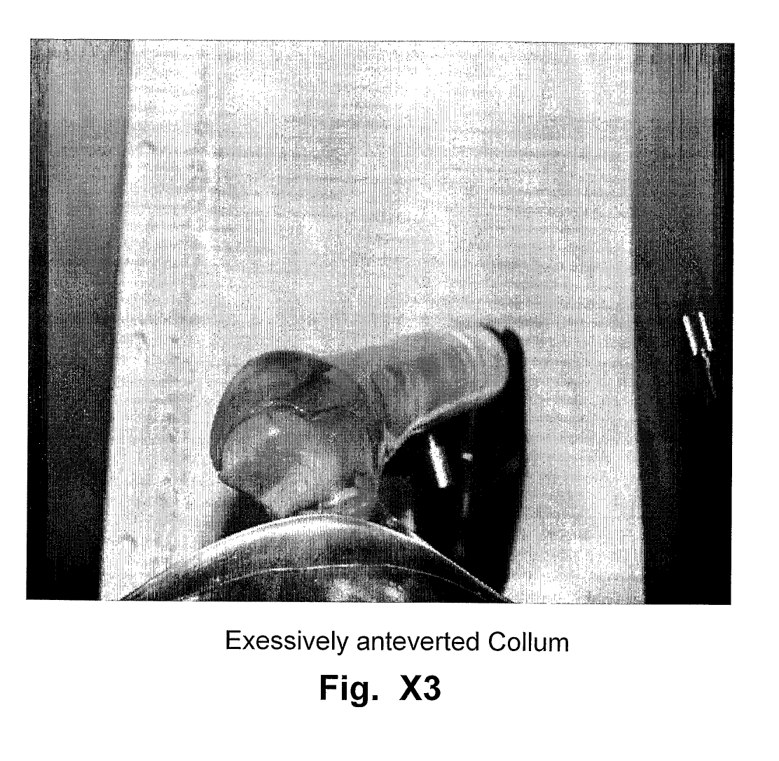

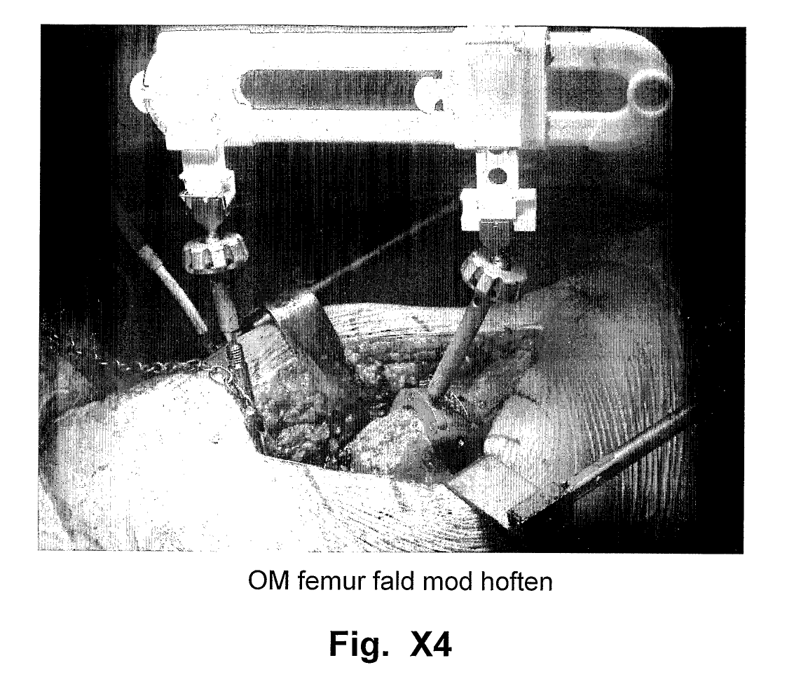

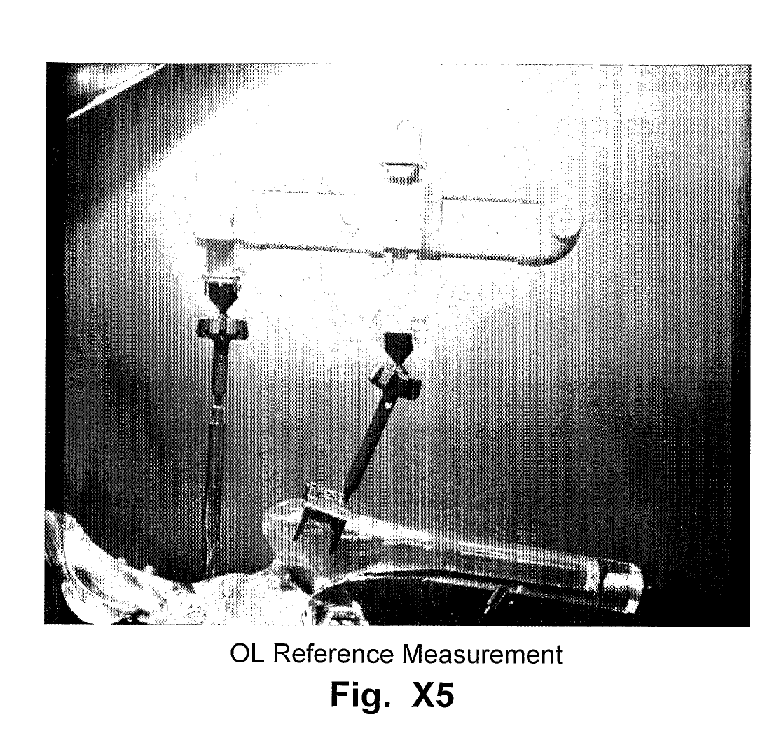

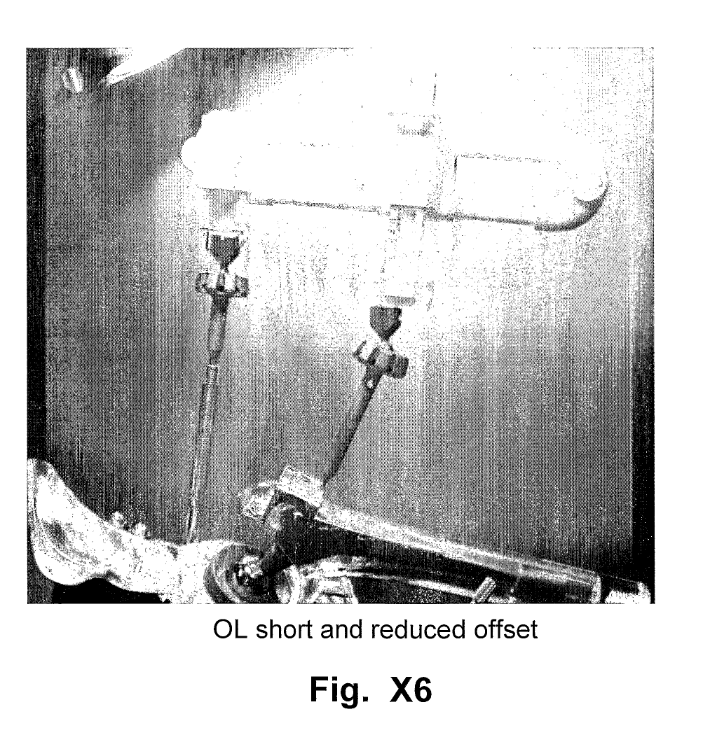

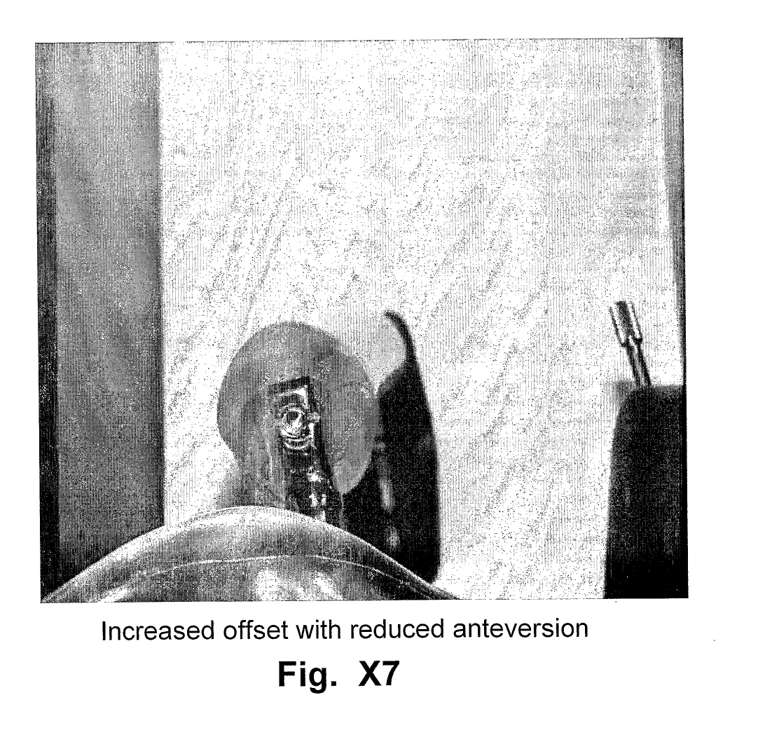

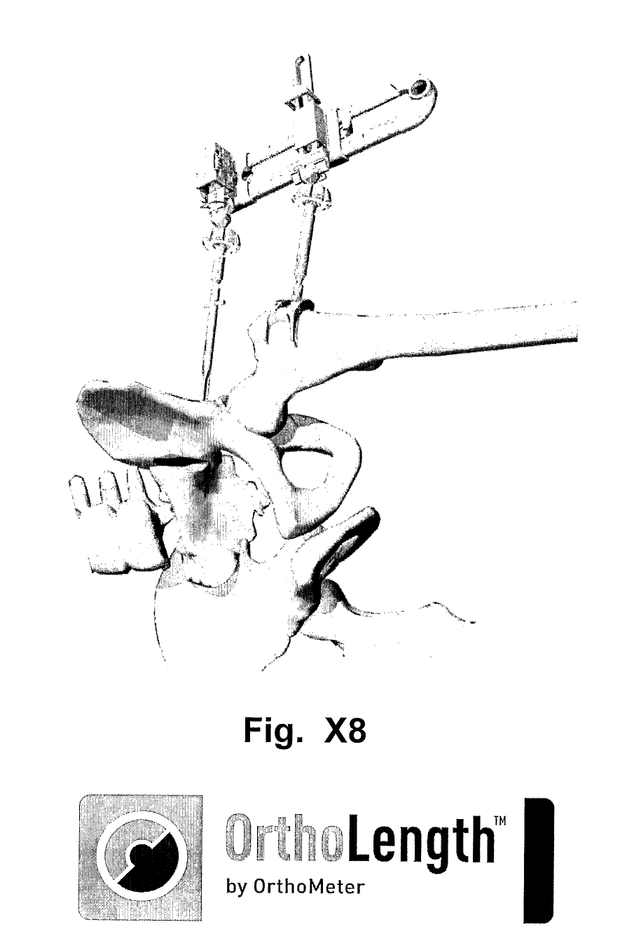

FIG. 6A is similar to FIG. 5A, wherein the first and second arm are rotated in the opposite directing, as another example of the flexibility of adjustment to a fix point connected to the pelvis;

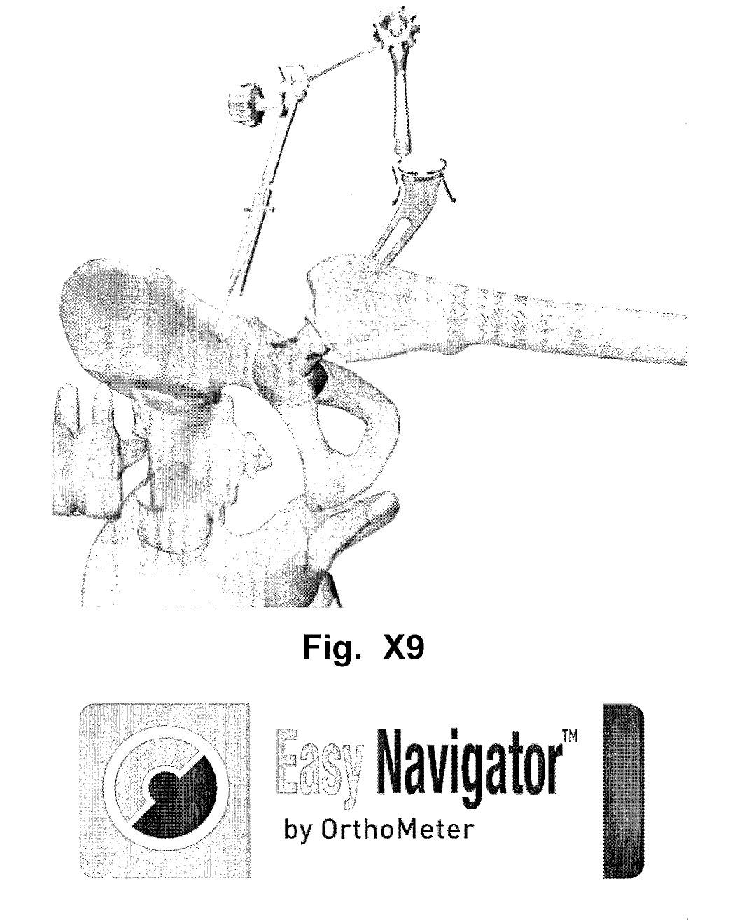

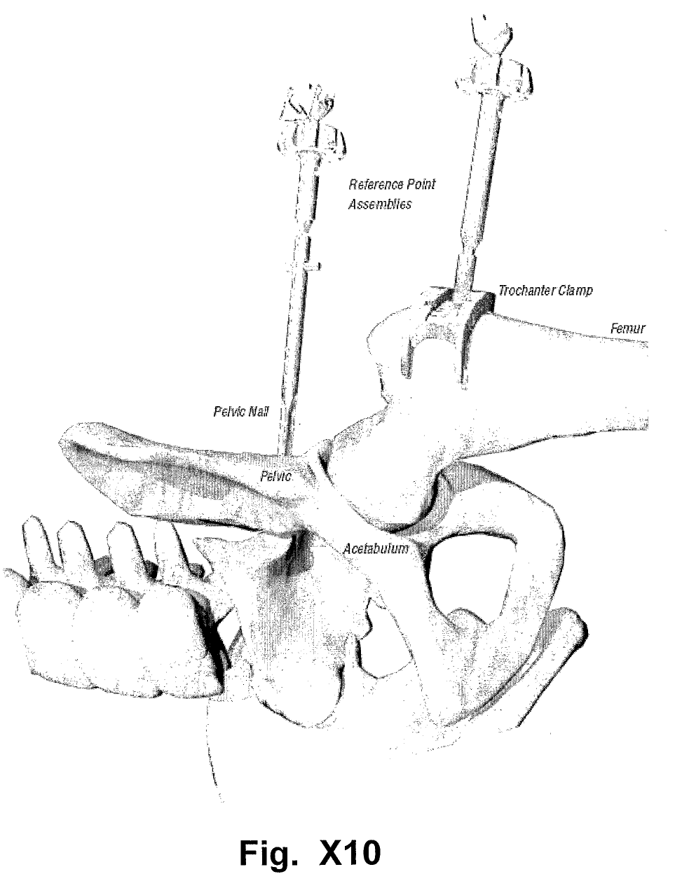

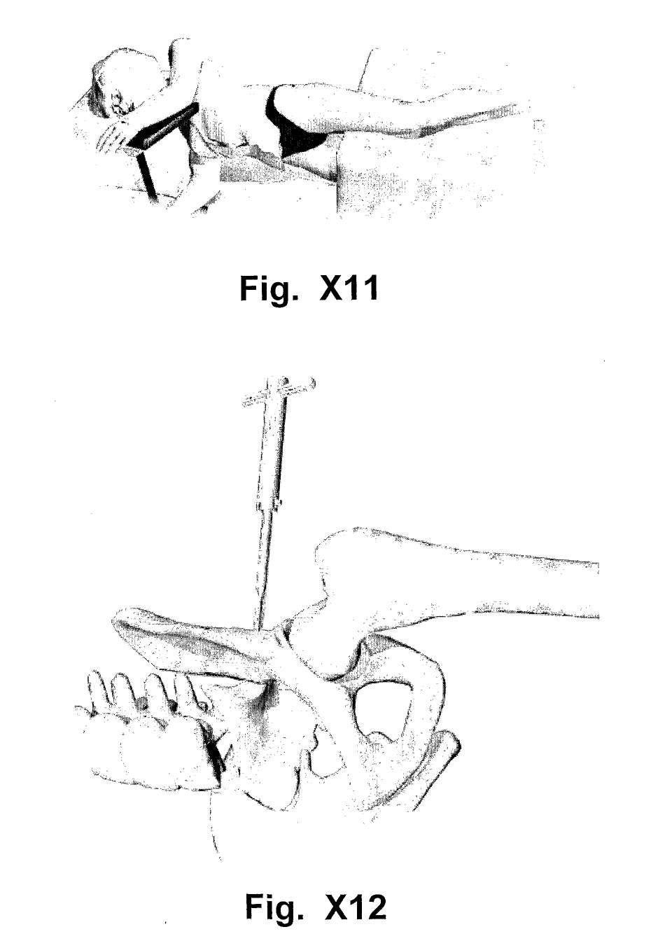

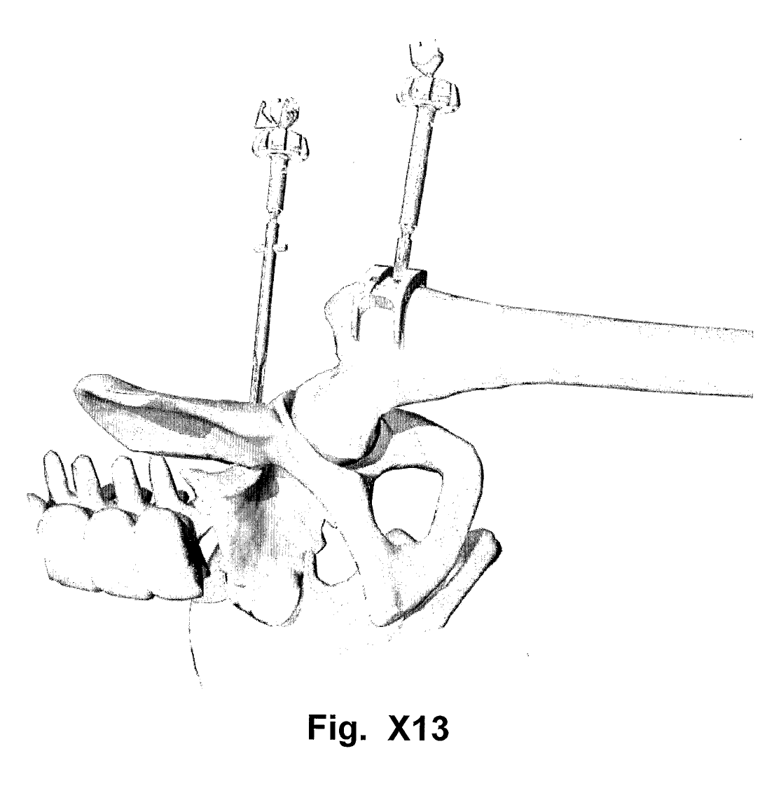

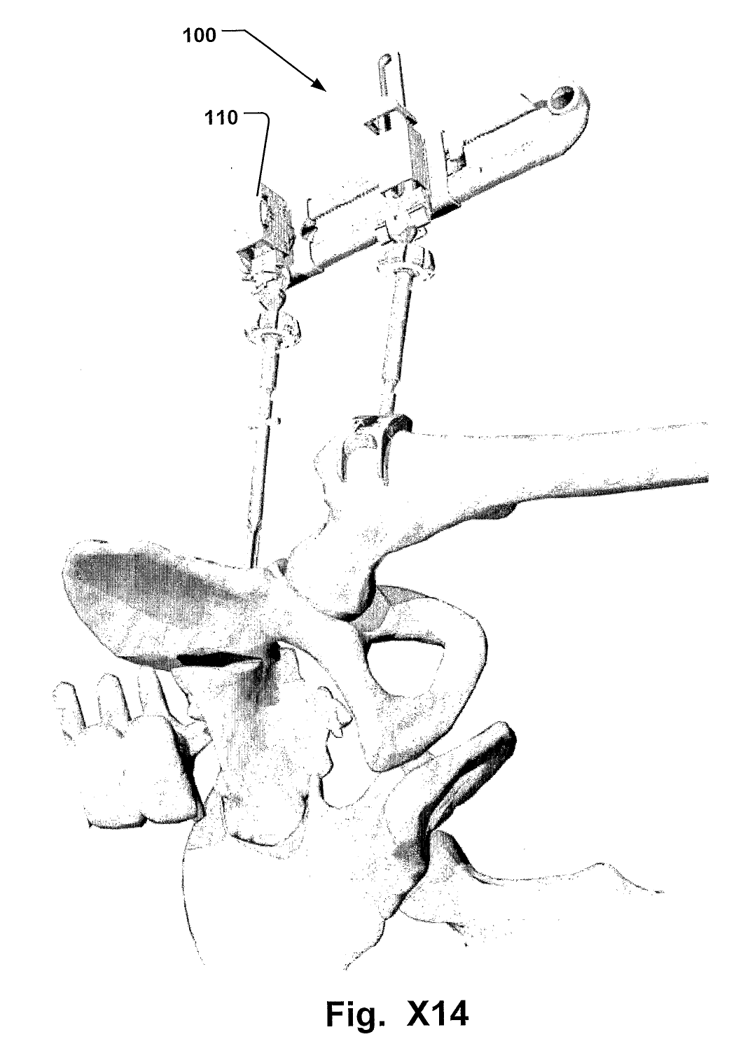

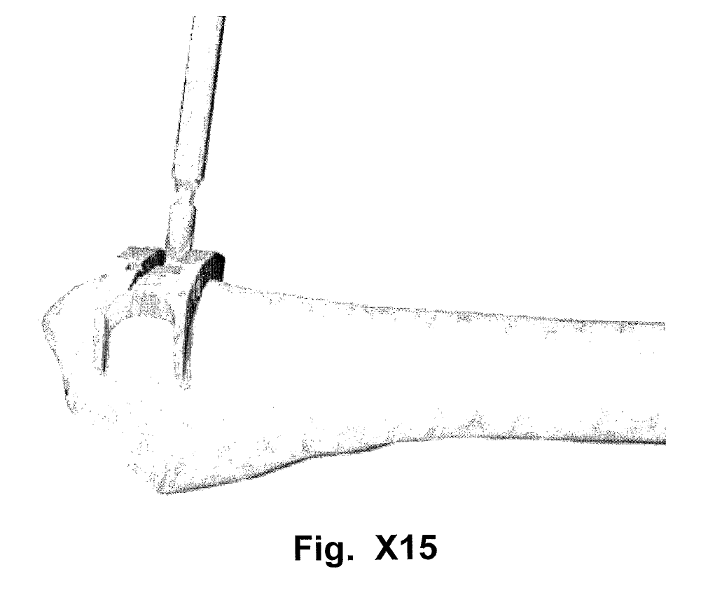

FIG. 7A shows the back of the tool of FIGS. 1A to 6A;

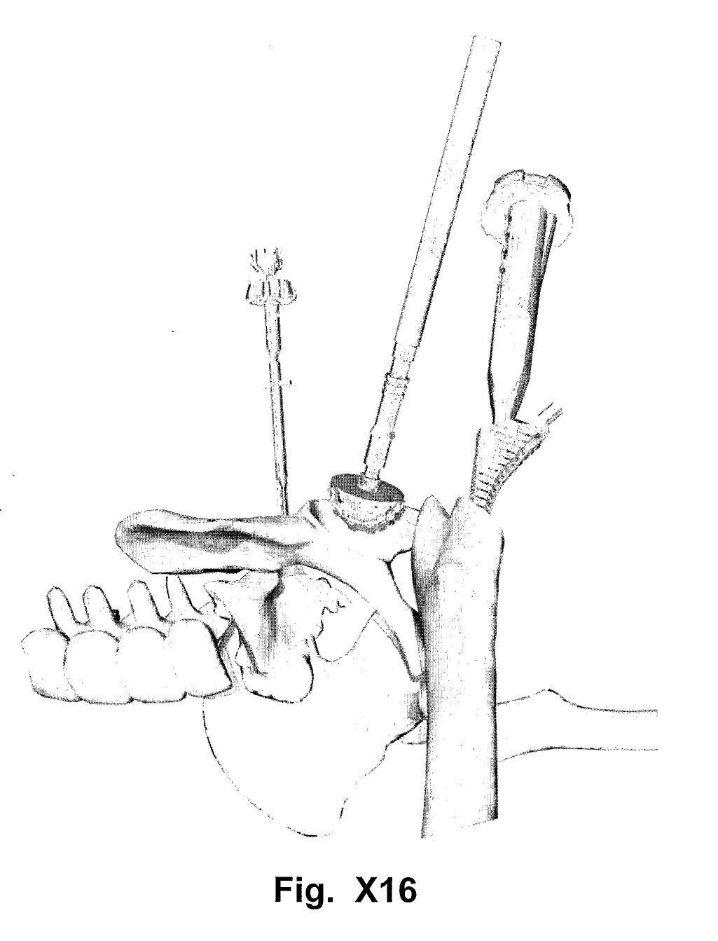

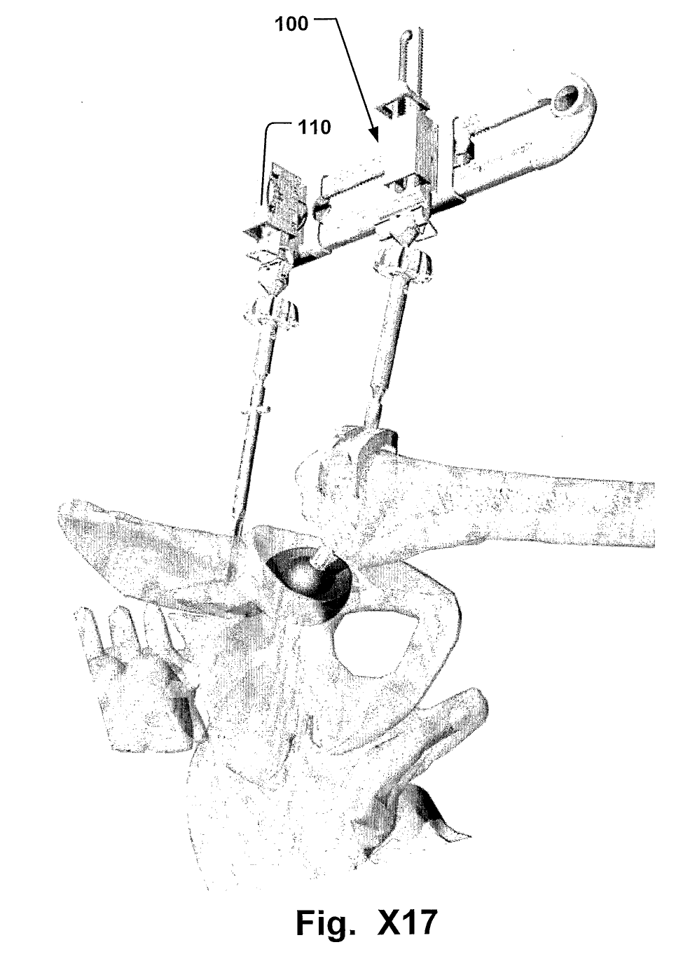

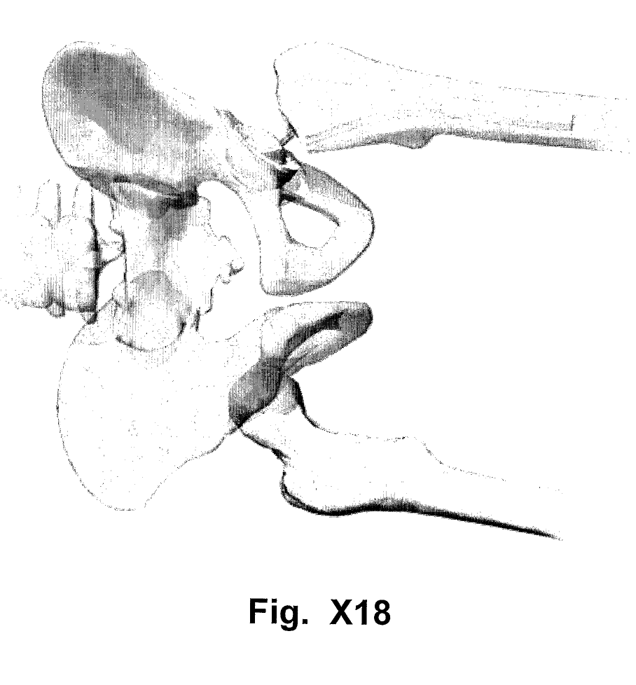

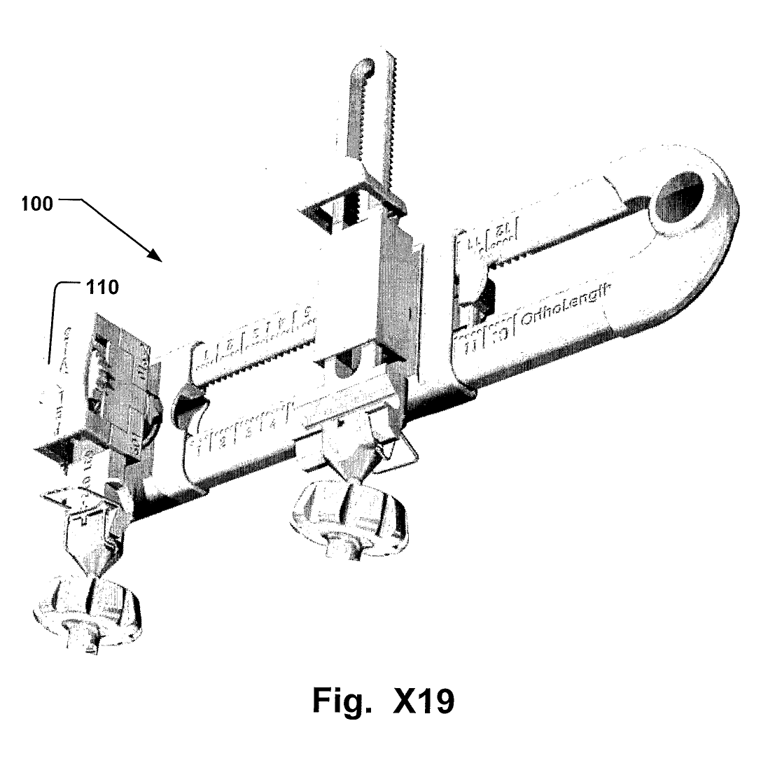

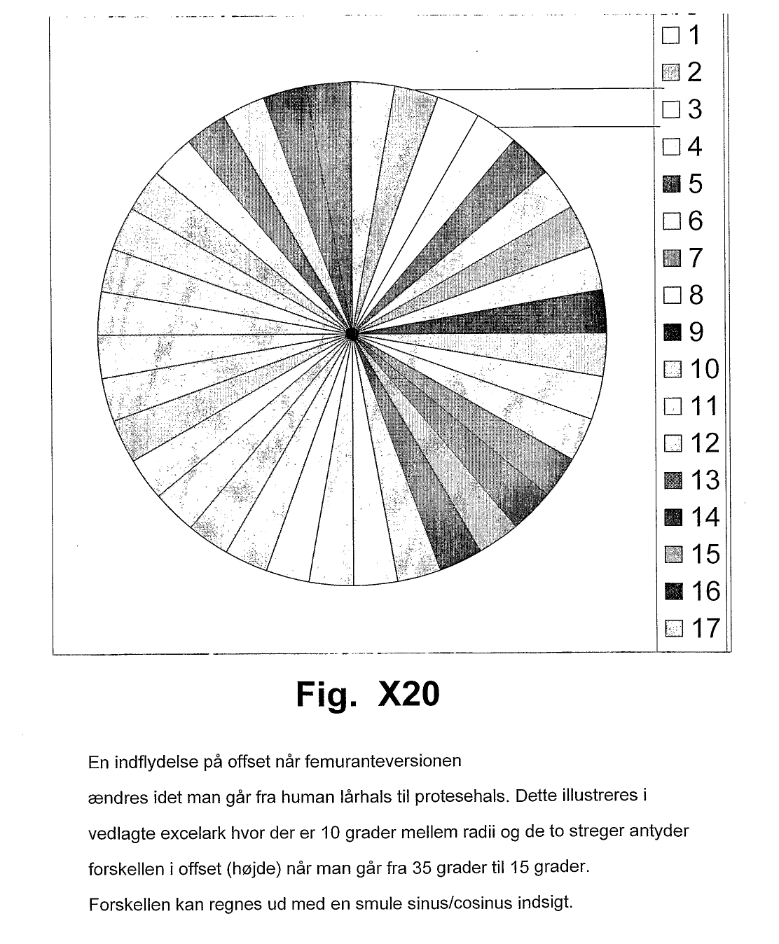

FIG. 8A shows a detail of the back, attachment point and bore of the second arm,

FIG. 9 is a perspective view of on of the ends of the main body of the tool, including a bore; also the second arm attachment, locking and rotating means is shown;

FIG. 10 shows a detail of the attachment, locking and rotating means of the second arm, the scales for measurements are evident;

FIG. 11 shows the second arm moved along the longitudinal axis of the main body;

FIG. 12 shows a detail of the attachment, locking and rotating means of the first arm, the scales for measurements are evident;

FIGS. 13A-13E show different connection members arranged at one of the ends of said first and second arm, where said connection members are adapted to interact with receptors at the supports; wherein the arm of FIGS. 13B and 13C has a notch for fixing the position of the connection member in a corresponding receptor, and the arm of FIGS. 13D and 13E has an advantageous groove for fast locking by spring loading;

FIG. 14 shows a perspective view of an embodiment of the measurement tool;

FIG. 15 shows a fixating system of an arm according to one embodiment of the present invention;

FIGS. 16 and 17 illustrate how the measurement tool is positioned into supports;

FIG. 18 shows the attachment of the supports to pelvis and femur respectively;

FIG. 19 to 26 illustrate the use of the measurement tool according to an embodiment of the invention;

FIG. 27 illustrates the measurement tool according to an embodiment of the invention in detail, attached to corresponding supports;

FIG. 28 illustrates an embodiment of the connection members and/or receptors of first and second arms and supports, respectively; and

Figs. X1-X20 illustrate a measurement tool according to an embodiment of the present invention.

### DETAILED DESCRIPTION OF EMBODIMENTS

Proper control over leg length and offset during total hip replacement can only obtained through comparison of a series of repeated measurements.

1) The first measurement is performed as a combination of measurements on preoperative x-rays and clinical measurements on the patient performed in the outpatient clinic when the surgery is planned.

2) The next measurement is performed between a pelvic and a femoral landmarks performed in the early stage of the surgery, before the femoral neck is divided. For full control of the procedure, two more measurements should be carried out,

3) One measurement should be carried out during a trial reduction of the joint with temporary prosthesis components and

4) One when the actual components have been inserted still though, with a choice between different neck lengths.

A series of measurements as described above enables the surgeon to choose prosthesis components (stage 3 to 4) to achieve the measurements that were planned between stages 1 and 2, and to further finally adjust the interface at step 4.

An obvious requirement for reliability is reproducible position of the patient on the operation table during stages 2 through to 4. Such reproducibility is not a given, because considerable force exerted by the surgeon during dislocation inevitably will shift the position of the patient. Also, the hip and knee is flexed 90 degrees and the hip rotated 90 degrees during dislocation and reduction, making it highly unlikely that the leg ends up in the same position between stages.

Several areas are amended for reliable function of the measurement device shown in FIGS. 1 to 28, by the measurement device shown in Figs. X1 to X20:

A. Reliable and reproducible positioning of the patient are based on reference points, e.g. landmarks, which are clearly defined by lying on a longitudinal axis through the reference points, thereby re-establishing the same relative orientation between the reference point on the pelvis and the one on the femur. This longitudinal axis, is spatially well defined and the Measuring Points that engages the Reference Points provide sufficient information to determine whether the same axis has been achieved during measurements 3 and 4 as during 1. Consequently, the present device aiming at yielding reliable measurements is based on measuring points engaging reference points over a contact area long enough to effectively demonstrate whether parallelism has been achieved.

B. In most individuals, the anteversion of the human femoral neck varies between 10 and 20 degrees but considerable greater anteversion is regularly encountered during surgery. Irrespective of original anatomy, the surgeon will aim for the a degree of anteversion of the prosthesis neck appropriate for a prosthetic joint, averaging 15-20 degrees, as re-establishing excessive anteversion will render patients prone to impingement and dislocations. Through reducing anteversion between stages 2 and 3, the femoral reference point is shifted posteriorly to be lying on a long axis parallel but behind the first one as established during the measurement in stage 2. This is illustrated for instance in Fig. X1-X7 and X20. Consequently, the device provides reliable measurements and features the capability to shift one of the anteriorly/posteriorly along an axis orthogonal to the longitudinal axis of the measuring device, comparable to as one would otherwise be measuring along the hypotenuse of a right angle triangle.

C. In cases where a greater degree of anteversion is reduced when the prosthetic joint is mounted—and the necklength is kept unchanged (e.g. if a patient with 35 degrees of femoral neck anteversion receives a prosthesis with the trunnion in 15 degrees of anteversion) the resultant offset is inadvertently increased as the greater trochanter is rotated laterally on a radius with the center of rotation in the prosthesis head. To a certain degree this lateralisation may be demonstrated by internally rotating the hip joint. It should be noted, though, that this latter lateralisation will involve a real rotation, whereas the lateralisation seen as a result of prosthetic replacement, will have the effect of a parallel shift anteriorly of the greater trochanter, since the femoral shaft will be “rotated” around the prosthesis shaft. It can be calculated that with a prosthesis offset of 40 mm, the increase in offset resulting from reducing anteversion from 35 degrees to 15 degrees is approximately 6.3 mm, which, from a biomechanical point of view is significant.

Consequently, the device 100 provides reliable measurements and features the capability to shift one of the anteriorly/posteriorly along an axis orthogonal to the longitudinal axis of the measuring device by means of an adjustment means 110, as shown in Fig. X19.

Adjustment means 110 allows for compensation orthogonal to the longitudinal axis of the measuring device 100. Adjustment means 110 allows also for reading the orthogonal offset, e.g. for determining if the offset is within certain desired physiological limits in order to ensure a correct biomechanics of the hip joint after replacement.

In FIG. 15 a fixation system of an arm on an adjustment means 110 is disclosed. In this system a turning knob is used to fixate the position of the arm. The fixation system according to FIG. 15 may provide an easier way of fixating the arm in a preferred position, since the measuring device may be held in position by the hands of the user, while only the thumb of one hand needs to be used to fixate the desired position of said adjustment means.

FIG. 1 illustrates a positioning tool 1 according to one embodiment of the present invention. The positioning tool 1 comprises two supports 2, 2′, a handle part 14 and two flexible arms 7, 7′. The supports 2,2′ are fastened in a conventional way to the femur and the pelvis, respectively, by means of screws 3, 3′. The screws may alternatively be substituted by pins. To avoid or reduce damage to soft tissue a protective sleeve 4,4′ are placed around the part of the screws that are in contact with soft tissue. An alternative method for fastening the supports is by means of clamps that are clamped to a bone.

The handle part 14 comprises a stem 16 and a fork-member 15. The handle part 14 substitutes the handle in the tool 30 according to WO 01/19296 (anteversion head), wherein the fork portion is designed for interaction with the anteversion head 30 as described in the mentioned publication.

The fork member 15 is connected to the stem by means of a ball joint 17. The ball joint 17 may be locked in a wanted angle by means of a not shown push rod inside the stem 16 controlled by a not shown screw in the opposite end of the stem relative to the ball joint 17. When the push rod is pushed against the ball in the ball joint, the position of the fork member 15 relative to the stem becomes temporarily locked. In an alternative embodiment the fork member 15 and stem 16 are fixed relative to each other. A tool set may then include 2 or more combined fork and stem units having different angle between the parts so that the surgeon may choose the unit having the appropriate angle.

At the opposite end of the stem relative to the ball joint 17, the stem is provided with means to temporarily and removable connect the stem 16 to the flexible arms 7, 7′. The means to temporarily and removable connect the stem to the flexible arms are in the illustrated embodiment bores (not shown) at the end of the stem designed to interact with corresponding pins 13 at a connection plate 12,12′ on the arms 7,7′. The pins 13 and the bores are designed so that the position of the arms relative to the stem are unambiguously defined when the pins are in the bores. The pins at the first arm 7 are also different from the pins at the second arm 7′, either in relative position or in shape or dimension of the pins, so that it is impossible to mix up the arms.

Connection members 6,6′ are provided at the opposite end of the flexible arms 7,7′.

The connection members 6,6′ are designed to fit into receptors 5,5′ at the top of the supports 2,2′. The receptors 5,5′ are formations that can interact with the connection members, such as grooves, ridges or other suitable formations. In the preferred embodiment, the receptors 5,5′ are grooves at the top of the supports 2,2′. According to one alternative embodiment one of or both receptors may be provided with a graded scale in one or more directions. This may be obtained, e.g. by using a ball shaped receptor having graded scales. This allows the surgeon to adjust the direction of the flexible arms in a controlled way, if necessary.

Each of the illustrated flexible arms 7,7′ comprise a first 8,8′ and a second 9,9′ arm member. The arm members are rotary connected in a rotary link 10,10′, which is rotary about an axis perpendicular to the longitudinal axis of arm members, close to one end of the arm members. At their free end, the arm members 8,8′, 9,9′ are connected to the connection plates 12,12′ and the connection members 6,6′ respectively, by means of ball joints 20,20′, 21,21′.

The rotary links 10,10′ are adjustable by means of control wheels 11,11′. FIG. 3 shows the section B-B of the first arm member 8 and the rotary link 10 in FIG. 1.

The rotary link comprises a bolt 28 having a longitudinal axis coinciding with the axis of rotation of the rotary link. The bolt 28 runs through both arm members close to one end and mainly perpendicular to their longitudinal axis.

A push rod 25 is provided longitudinally inside the arm member 8 and rests against a ball 26 in the ball joint 21 at one end and against a conical body 27 in the rotary link 10.

In the embodiment illustrated in FIG. 3 the conical body 27 is integrated in the control wheel 11, whereas the conical body 27 illustrated in FIG. 4, is a separate part. The control wheel 11 is screwed onto a bolt 28. The bolt 28 has a conical head 29.

Alternatively may a conical body substitute the conical head 29.

When turning the control wheel the bolt one way the conical head and the conical body are pressed towards each other or removed from each other depending on the direction of rotation. When the control wheel is tightened, i.e. that the conical body and the conical head are forced towards each other, the conical body 27 is forced towards the push rod 25. The pushrod is then forced against the ball 26 of the ball joint 21 resulting in locking of the ball joint. At the same time the conical head of the bolt is forced towards a corresponding push rod 24 inside the second arm member 9 resting against a ball in the ball joint 20. Thus, the tightening of the control wheel 11 results in locking of the ball joints 20,21 and the rotational joint between the first arm member 8 and the second arm member 9 so that the arm 7 is fixed in a given configuration.

The joints 10,10′, 20,20′, 21,21′ may be locked by other means than described above. The rotational links 10,10′ may be locked as described using a wheel and a bolt to tighten the links. The ball joints 20,20′, 21,21′ might be locked by turning nuts that tightens and locks joint. The ball joints might also be locked by means of a lever operating an eccentric hinge exercising a force directly or indirectly to the balls of the ball joints to lock the ball in a wanted position.

The arms 7,7′ may also be substituted by telescopically adjustable substantially straight arms. The mechanism for the telescopic adjustment is not vital as long as it is possible to lock the arm at a wanted length. The skilled man in the art is aware of different ways to lock a telescopically adjustable arm.

The tool 1 is preferably used in combination with a measuring device 40. The measuring device 40 comprises a lengthy main body 41, an adjustable member 42 and an adjustable arm 43. Two bores 44,45, one close to one end of the main body 41 and the other in the adjustable member, may serve as templates for making bores for fastening the screws 3,3′ in femur and pelvis, respectively.

Connection members 46,47 at an arm 48 at the adjustable member 42, and at the adjustable arm 43, respectively, are designed to rest against the receptors 5,5′ at the supports 2,2′ that are connected to the screws 3, 3′.

The adjustable member 42 may be moved along the main body 41. The adjustable member 42 may be locked to the main body 41 by means of a locking member 49 that are forced into engagement with adjustable member 42 and the main body 41. In the illustrated embodiment the arm 48 is a part of the locking member 49. The adjustable member 42 and locking member 49 are fastened to each other by means of hooks 51 mounted on the adjustable member 42 that are forced into engagement with notches 52 in a receiving member 53 on the locking member 49. The hooks 51 are not available for the user so that it is not possible to remove the locking member after the device is locked without breaking the locking member or the adjustable member. When the locking member is in engagement with the adjustable member a number of teeth 54 on the locking member 49 are in engagement with corresponding teeth 55 on the main body 41 to lock the adjustable member to the main body in a given position.

The adjustable arm 43 is moveably mounted in a track 56 wherein the adjustable arm 43 may be moved along its longitudinal axis mainly perpendicular to the longitudinal axis of the main body and mainly parallel with the arm 48. The adjustable arm 43 may be locked in a wanted position by means of a strap 57. At one end the strap 57 is preferably hinged to the main body. When the adjustable arm is placed in the wanted position, the strap is forced against the adjustable arm so that not shown teeth or ribs at the strap interact with teeth 58 at the adjustable arm 43. The strap is locked by means of a not shown tongue that is forced into interaction with a not shown notch. It is preferred that the strap is fastened to the main body so that it is not lost before it is used to lock the adjustable arm.

The measuring device 40 is preferably made of a medically approved plastic material allowing the necessary stability and stiffness of the device. The device may then be sterilized and packed in a sterile package. As mentioned above, it is preferred that the locking of both the adjustable member and the adjustable arm is irreversible, meaning that the device is broken and not possible to use again if anybody tries to unlock the device.

The measuring device preferably disposable and is thrown after use, to avoid problems in cleaning and sterilization of a used device.

### Surgical Procedure

The normal procedure for insertion of an artificial hip joint using the present tool and system is as follows, without being bound of the described sequence of the procedural steps:

1) The patient is examined and a plan for any adjustments of length of the limb, i.e. the length between the floor and hip joint, and offset, i.e. the distance between the longitudinal axis of the femur and the sagittal plane of the body, is made preferably when the patient visits the out patient clinic for preoperative examination.

2) After preparation for surgery the patient is placed on the operating table in a defined “start position”, e.g. positioned on the side with the relevant hip up and the legs parallel at the operating table and is supported and stabilised so that the patients trunk and the leg facing down are kept in the same position during the surgical procedure.

3) Stab incisions are made to get access to the femur and pelvis to make bores for fastening the screws 3, 3′ and the supports 2, 2′. The measuring device 40 is preferably used as a template for making the bores in the femur and the pelvis.

The adjustable body 42 is placed at a predetermined distance from the bore 44 taking into account the length required for the prosthesis in question. Then the bores into the femur and pelvis are made using the bores 44 and 45 as drill guide. After making the bores in the pelvis and femur, the screws 3, 3′ are entered into the bores using bores 44 and 45 at the measuring device as guides to ensure that the bores in the bones are substantially parallel. Preferably protective sleeves 4,4′ are put onto the screw to avoid damage to soft tissue due to contact between the screw and the soft tissue before the supports 2,2′ are connected to the screws.

4) The measuring device 40 is then used to measure the distance in three dimensions between the supports 2, 2′. The distance is measured by placing a connecting member 46 at the end of the arm 48, is put into the receptor 5 at the top of the supports 2 and the support is rotated, if necessary, to align the receptor relative to the other support 2′. The adjustable member 42 and the adjustable arm 43 are then adjusted to allow a connection member 47 at the adjustable arm 43 to be put into the receptor 5′ at the other support 2′. If necessary the other support 2′ is rotated to align the receptor 5′. After placing both the connection members 46,47 into the receptor 5,5′ and assuring that the connection members both are in full contact with the receptor, the position of the adjustable body at the main body 41 and the position of the adjustable arm relative to the main body 41 are read and registered. Any planned adjustment in the length of the limb or offset is then made by adjusting the position of the adjustable body or the adjustable arm, before the adjustable arm and the adjustable body are locked to the main body.

5) The necessary incisions for performing the total hip replacement are then made.

6) The neck of femur is divided and the head of femur is removed. The internal femoral canal is then prepared by rasps to receive the stem 31 of the artificial hip joint and the pelvic cavity is hollowed to receive the cup 32 of the artificial hip joint.

7) The rasp or a prosthesis stem 31 is temporarily inserted into the prepared femoral canal. An anteversion head 30 is mounted on the prosthesis stem as illustrated in FIG. 1 and as described in WO 01/19296. A provisional prosthesis head 33 and a collar 34 are put on the prosthesis neck as an elongation of prosthesis stem 31. The collar 34 and the preliminary head 33 may alternatively be made in one piece and the size of the head may be so large to act as a spacer thus replacing the cup. When a cup is used, the function of the preliminary head and the collar is to interact with the prosthesis cup in that the head rests in a recess in the cup and the collar rests against the surface connecting the recess and the outer surface of the cup, to define the angle between the cup and the prosthesis stem.

The prosthesis head 33 and collar 34 are connected to the handle part 14 of the positioning tool 1 by means of the fork member 15 comprising two guide rods 35 that are inserted into guide holes in the collar and/or the guide head. The fork may also be directly connected to the prosthesis, the rasp or extensions thereof.

8) A prosthesis cup is placed into the prepared recess in the pelvis and the artificial joint is put together. The patient is again placed in the start position as during the measurement under the above item 4. The connecting member 46 at the arm 48 is again put into the receptor 5 at the support 2 and the mutual position of the supports is re-established by adjusting the leg of the patient until the connecting member 47 at the adjustable arm 43 rests in the receptor 6 at the other support 2′. The measuring device is then removed and the flexible arms 7,7′ are connected to the handle part 14 as described above. The flexible arms 7,7′ and the joints 20,20′, 21,21′, 10 and 10′ are adjusted so that the arms are connected to the handle part at the same time as the connection members 6,6′ are resting in the receptor 5,5′. The control wheel 11,11′ is then tightened so that the flexible arms and the joints are locked in this position. The arms 7,7′ are then removed and the artificial joint is disassembled.

9) The anteversion head is connected to the cup 32 of the prosthesis and the handle part 14 is connected to the anteversion head as described above before the cup is placed in the prepared pelvic cavity together with cement to fasten the cup. The other flexible arm not used under step 9) above, is fastened to the top of the handle part as described above and the position of the cup is adjusted until the connection member 6′ of the flexible arm 7′ rests in the longitudinal axis 5′ of the support 2′ fastened to pelvis. After recreating the position of the cup relative to the support as under item 8) above, in this way, the cup is held in this position until the cement is hardened sufficiently to remove the anteversion head.

10) The prosthesis stem 31 is cemented into the prepared femoral canal. The position of the prosthesis stem in the femur is controlled by connecting the anteversion head 30 to the prosthesis stem, connecting the handle part to the anteversion head (or to the prosthesis or extensions to the prosthesis) as described above, connecting the arm 7,7′ that was used to measure the distance between the top of the handle part and the support on femur, to the top of the handle part and adjusting the position of the prosthesis stem 31 until the connecting member 6 is resting in the receptor. After recreating the position of the prosthesis stem relative to the support as under step 8) above in this way, the position is held until the cement is hardened sufficiently to remove the anteversion head.

11) After replacing the head 33 of the anteversion head with a permanent head for the prosthesis, the leg of the patient is again moved into the basic position to assemble the artificial joint and the surgery is finished in the normal way and the supports and screws are removed.

The prosthesis stem used for provisional insertion in the present description and claims, may be the prosthesis stem that are to be placed permanently into the femur of the patient, it may be a provisional prosthesis stem only used for provisional insertions and measurements or it may be the rasp used for preparing the hollow in the femur for insertion of the prosthesis.

The term start position used in the present description and claims may any position that is useful for performing the surgery and that is easy to control and reproduce. The start position may be with the patient on the side as described above or a position where the patient is lying like a tin soldier having the toes pointing upwards. Additional tools, that are traditionally used to align the patient and control the position, may be used to control and reproduce the start position. The man skilled in the art will recognize which position that is the best suitable start position for a given situation and which tools and techniques to use with regard to surgery and the positioning of the patient.

The surgical procedure described above is the preferred surgical procedure as it allows maximum control even in minimally invasive surgery where the surgeon is performing surgery through minimal incisions and where extra tools are needed to position the prosthesis correctly.

### Alternative Surgical Procedure I

During this procedure, an acceptable positioning of the parts may be obtained without using the measuring device 40. This procedure comprises the following steps:

1) The patient is examined and a plan for surgery is made, preferably in the out patient clinic.

2) After preparation for surgery the patient is placed at the operating table in a defined “start position”, e.g. lying on the side the hip to be operated superior and the legs parallel at the operating table supported and stabilised so that the patients trunk and the leg facing down are kept in the same position during the surgical procedure.

3) Stab incisions are made to get access to the femur and pelvis to make drill canals for fastening the screws 3,3′ and the supports 2, 2′. The drill canals into pelvis and femur are made. Preferably a template is used both for the drill when making the drill canals and when fastening the screws to ensure that the drill canals in the bones and subsequently the screws are substantially parallel.

Preferably protective sleeves 4,4′ are put onto the screw to avoid damage to soft tissue due to contact between the screw and the soft tissue before the supports 2,2′ are connected to the screws.

4) The incisions required for total hip replacement, are then made.

5) The neck of femur is divided and the head of femur is removed. The femoral canal is prepared to receive the stem 31 of the artificial hip joint and the pelvic cavity is prepared to receive the cup 32 of the artificial hip joint.

6) A prosthesis stem 31 is temporarily inserted into the femoral canal. An anteversion head 30 is mounted on the prosthesis stem as illustrated in FIG. 1 and as described in WO 01/19296. A provisional prosthesis head 33 and a collar 34 are put on the prosthesis neck as an elongation of prosthesis stem 31. The collar 34 and the preliminary head 33 may alternatively be made in one piece.

The function of the preliminary head and the collar are to interact with the prosthesis cup in that the head rests in a recess in the cup and the collar rests against the surface connecting the recess and the outer surface of the cup, to define the angle between the cup and the prosthesis stem. In an alternative design, the preliminary head has an outer diameter equal to the cup, thus replacing the cup as a spacer.

The prosthesis head 33 and collar 34 are connected to the handle part 14 of the positioning tool 1 by means of the fork member 15 comprising two guide rods 35 that are inserted into guide holes in the collar and/or the guide head.

7) A prosthesis cup is placed into the prepared recess in the pelvis and the artificial joint is put together. The patient is again placed in the start position and the surgeon ensures by visual inspection that the parts of the prosthesis are in place in the hollow in femur and pelvis, respectively. Thereafter it is ensured by means of the anteversion head that the mutual angle between the parts of the prosthesis is correct. The flexible arms 7,7′ are then connected to the handle part 14 as described above. The flexible arms 7,7′ and the joints 20,20′, 21,21′, 10 and 10′ are adjusted so that the arms are connected to the handle part at the same time as the connection members 6,6′ are resting in the receptors 5,5′. The control wheel 11,11′ is then tightened so that the flexible arms and the joints are locked in this position. The arms 7,7′ are then removed and the artificial joint is disassembled.

8) The anteversion head is mounted on an insertion tool (not shown) and connected to the cup 32 of the prosthesis and the handle part 14 is connected to the anteversion head as described above before the cup is placed in the prepared pelvic cavity together with cement to fasten the cup or without cement for force fit according to the choice of prosthesis. The flexible arm 7,7′ is fastened to the top of the handle part as described above and the position of the cup is adjusted until the connection member 6′ of the flexible arm 7′ rests in the receptor 5′ of the support 2′ fastened to pelvis. After recreating the position of the cup relative to the support as under item 8) above, in this way, the cup is held in this position until durable fixation as been obtained e.g. by the cement having hardened sufficiently for the anteversion head to be removed.

9) The prosthesis stem 31 is cemented into the hollowed femur. The position of the prosthesis stem in the femur is controlled by connecting the anteversion head 30 to the prosthesis stem, connecting the handle part to the anteversion head as described above, connecting the other flexible arm 7,7′ not used under step 8) above that was used to measure the distance between the top of the handle part and the support at femur, to the top of the handle part and adjusting the position of the prosthesis stem 31 until the connecting member 6 is resting in the receptor. After recreating the position of the prosthesis stem relative to the support as under step 8) above in this way, the position is held until the cement is hardened sufficiently to remove the anteversion head.

10) After replacing the head 33 of the anteversion head with a permanent head for the prosthesis, the leg of the patient is again moved into the start position to assemble the artificial joint and the surgery is finished in the normal way and the supports and screws are removed.

### Alternative Surgical Procedure II

During this procedure, an acceptable positioning of the parts may be obtained without using the positioning tool 1. This procedure comprises the following steps:

1) The patient is examined and a plan for any adjustments of length of the limb, i.e. the length between the knee and hip joint, and offset, i.e. the distance between the longitudinal axis of the femur and the sagittal plane of the body, is made preferably in the out patient clinic.

2) After preparation for surgery the patient is placed at the operating table in a defined “start position”, e.g. lying on the side with the hip to be operated up and the legs parallel at the operating table and supported and stabilised so that the patients trunk and the leg facing down are kept in the same position during the surgical procedure.

3) Stab incisions are made to get access to the femur and pelvis to make drill canals for fastening the screws 3,3′ and the supports 2,2′. The measuring device 40 is preferably used as a template for making the drill canals in the femur and the pelvis. The adjustable body 42 is placed at a predetermined distance from the drill guide canal 44. Then the drill canals into the femur and pelvis are made using the bores 44 and 45 as drill guides. After making the bores in the pelvis and femur, the screws 3,3′ are entered into the bores using bores 44 and 45 at the measuring device as guides to ensure that the screws in the bones are substantially parallel. Preferably protective sleeves 4,4′ are put onto the screw to avoid damage to soft tissue due to contact between the screw and the soft tissue before the supports 2,2′ are connected to the screws.

4) The measuring device 40 is then used to measure the distance in two dimensions between the supports 2,2′. In addition to the distance in two dimensions, the alignment of the supports and ensures correct position also in the third dimension. The distance is measured by placing a connecting member 46 at the end of the arm 48, into the receptor 5 at the top of the supports 2 and the support is rotated, if necessary, to align the receptor relative to the other support 2′. The adjustable member 42 and the adjustable arm 43 are then adjusted to allow a connection member 47 at the adjustable arm 43 to be put into the receptor 5′ at the other support 2′. If necessary the other support 2′ is rotated to align the receptor 5′. After placing both the connection members 46,47 into the receptors 5,5′ and assuring that the connection members both are in full contact with the receptors, the position of the adjustable body at the main body 41 and the position of the adjustable arm relative to the main body 41 are read and registered. Any planned adjustment in the length of the limb or offset is then made by adjusting the position of the adjustable body or the adjustable arm, before the adjustable arm and the adjustable body are locked to the main body.

5) The surgical incisions for the total hip replacement, are then made.

6) The neck of femur is divided and the head of femur is removed. The femur is then hollowed to receive the stem 31 of the artificial hip joint and the pelvic cavity is hollowed to receive the cup 32 of the artificial hip joint.

7) Cement is put into the pelvic recess and a prosthesis cup is placed into the recess in the pelvis and the artificial joint is put together. The patient is again placed in the basic position as during the measurement under the above item 4. The connecting member 46 at the arm 48 is again put into the receptor 5 at the support 2 and the mutual position of the supports is re-established by adjusting the leg of the patient until the connecting member 47 at the adjustable arm 43 rests in the receptor 6 at the other support 2′. The hip joint is then held in this position until the cement is hardened.

8) A prosthesis stem 31 is cemented (or force fitted) into the hollow femur. After curing of the cement, an anteversion head 30 is mounted on the prosthesis stem as illustrated in FIG. 1 and as described in WO 01/19296. A provisional prosthesis head 33 and a collar 34 are put on the prosthesis neck as an elongation of prosthesis stem 31. The collar 34 and the preliminary head 33 may alternatively be made in one piece. The function of the preliminary head and the collar are to interact with the prosthesis cup in that the head rests in a recess in the cup and the collar rests against the surface connecting the recess and the outer surface of the cup, to define the angle between the cup and the prosthesis stem.

The prosthesis head 33 and collar 34 are connected to the handle part 14 that substitutes the handle in the in the device according to WO 01/19296 wherein the fork member 15 comprising two guide rods 35 that inserted into guide holes in the collar and/or the guide head.

9) After replacing the head 33 of the anteversion head with a permanent head for the prosthesis, the leg of the patient is again moved into the start position to assemble the artificial joint and the surgery is finished in the normal way and the supports and screws are removed.

Above, the invention is described with reference to the presently preferred embodiments of the system, tools and method, and relating to implantation of an artificial hip joint.

The present measuring devices may, however, also be used during other surgical procedures, such as implantation of artificial hinged joints or ball joints, such as an artificial knee prosthesis and other joints.

During control measurements, the prosthesis cup may be substituted by a spacer filling out a space in acetebulum corresponding to the space occupied by the cup.

The prosthesis stem described above has a prosthesis neck fixed to the stem. Alternatively a prosthesis stem having a prosthesis neck that is adjustably fixed to the stem, may be used. In using this type of prosthesis stem. The corrections described above for adjusting the position of the prosthesis stem before fixation, may be performed by adjusting the prosthesis neck after fixation of the stem.

FIG. 27 illustrates the measurement tool in detail, attached to corresponding supports, similar to an improved device 40.

The ref. Numerals in FIG. 27 are either three digit numbers, related to the tool or four digit numerals related to the fix points.

The measuring device of FIG. 27 comprises a lengthy main body 011-1, an adjustable member 006-1 and an adjustable arm. Two bores, one close to one end of the main body and the other in the adjustable member, may serve as templates for making bores for fastening screws or nails in the femur and pelvis, respectively.

Connection members (008-1) at an arm at the adjustable member, and at the adjustable arm, respectively, are designed to rest against the receptors at the supports 4170 that are connected to the screws or nails in the femur and pelvis, respectively.

The adjustable member may be moved along the main body. The adjustable member may be locked to the main body by means of a locking member that are forced into engagement with adjustable member and the main body. When the locking member is in engagement with the adjustable member a number of teeth on the locking member are in engagement with corresponding teeth on the main body to lock the adjustable member to the main body in a given position.

The adjustable arm is moveably mounted wherein the adjustable arm may be moved along its longitudinal axis and rotated as described above. The adjustable arm may be locked in a wanted position. When the adjustable arm is placed in the wanted position, a locking means is forced against the adjustable arm so that not shown teeth or ribs at the strap interact with teeth at the adjustable arm.

Hence, an advantageous attachment to screws or nails in the femur and pelvis, respectively, is achieved. The orientation of these screws or nails in the femur and pelvis, respectively, is no longer critical as the measurement tool is flexibly adaptable to any position of the screws or nails in the femur and pelvis, respectively.

FIG. 28 discloses an additional embodiment of the interaction between said supports 4170 and said connection members (008-1). In this embodiment said receptors on the supports 4170 and/or said connection members (008-1) are movable along sliding track. This sliding track may for example be curved to simplify the obtainment of perfect matching between the receptors on the support 4170 and the connection members (008-1). Such a sliding track may for example be arranged on said supports, whereupon the receptors are movable. It is also possible that said sliding track is arranged at the end of said first and/or second arm, upon which sliding track the connection members are movable/slidable. Preferably, the receptors and/or connection members may be fixated in a desired position on said sliding track, by for example a turning knob etc.

Figs. X1-X20 illustrates an improved measurement tool in detail, attached to corresponding supports, similar to the improved device 40 and the device shown in e.g. FIG. 26. The difference between these devices and the device 100 of Figs. X1-X20 is that adjusting means 110 allow for reliable measurements and features the capability to shift one of the anteriorly/posteriorly along an axis orthogonal to the longitudinal axis of the measuring device by means of an adjustment means 110, as shown in Fig. X19.

Especially Figs. X1 to X3 show that the movement orthogonally to the direction of the main body solves a problem of positioning the measuring device. It is clearly shown that it most probably will be an offset between the two supports, and thereby also the receptors, in a direction orthogonal to the direction of the measuring device. By providing at least one of said first and second arm with the possibility of moving in a plane, orthogonal to the plane of the main body of the measuring device, this problem may be solved. An illustration of this is for example shown in Fig. X1. Then the connection members, located in the end of said first and/or second arm may be fitted in the receptors on said supports, since said connection members are adapted to interact with receptors at said supports. Thus, the relation between said first and second plane anteriorly or posteriorly may be shifted along said second plane of the measuring device.

### Detailed Description of a Method and Use of the Improved Measurement Device:

Reference is particularly made to FIGS. 18 to 26, and X11-X18 describing steps 1 to 7 in these Figs. However, the remaining Figs. describing the improved measurement device are also to be considered.

### Pre-Operative Planning

Plan the adjustment of the offset and the leg length by measuring on X-rays and by measuring directly on the patient in the outpatient clinic.

Measurements must be written in the patient records for per-operative reference.

Positioning the Patient: “The Tin Soldier Position”

Essential for the OrthoLength™ technique is correct patient positioning on the operating table in a position resembling the standing position. We refer to this position as “The tin soldier position”. A special tunnel pillow may be supplied to support the leg to be operated on, while the contra lateral leg is supported with sand bags with both hip and knee extended.

It is recommended that the surgeon takes personal responsibility for positioning the patient, not leaving this important step to the assistant or scrub nurse.

Once the correct patient position has been obtained, care must be taken not to change patient position until Reference Points on pelvis and Femur are in place and correctly aligned.

### Positioning the Femural Reference Point (Trochanter Clamp):

The Femural Reference Point is mounted on the greater Trochanter.

Carefully identify the sciatic nerve. With the mono-legged side of the clamp, perforate the soft tissue and slide the mono-leg in along the posterior face of the greater trochanter, until its hocked tip engages the intertrochanteric crest.

Next, rest the clamp against the trochanter while tightening the clamp by screwing down the tightening screw. Make sure the double hocks sinks into the anterior aspect of the trochanter, and get a good grip.

### Positioning the Pelvic Reference Point:

When the joint capsule and the acetabular region are exposed, the Pelvic Nail is carefully positioned in the supra-acetabular region. Use the alignment holes on the OrthoLength main body to obtain the required axis-alignment between the Pelvic Reference Point and the Femural Reference Point.

First mount the Femural Reference Point Extension Rod on the Trochanter Clamp. Slide the hole on the OrthoLength Slider over the Extension Rod. Insert the Pelvic Nail in the Extractor Handle and slide the assembly through the appropriate hole at the distal part of the OrthoLength Main Body. Adjust Slider until the tip of the Pelvic Nail hits the desired supra acetabular position.

Before inserting the nail, the surgeon should identify the joint by digital palpation, paying attention to avoid placing the nail too close to—or—perforating the acetabular loft. Insert nail by using a mallet tapping on the Extraction T-Handle.

Remove Extractor Handle, remove OrthoLength.

To achieve perfect alignment, continue to adjust the rotation of the Reference Points until the keel-like Measuring Points of OrthoLength™ fits perfectly in the receiving grooves on the Reference Points.

### Measuring Leg Length and Offset (The Reference Measurement):

Intra-operative Leg Length and Offset is measured between the Pelvic and Femural Reference Points. The Reference Points are equipped with hexagonal adapters which fit in the Pelvic Nail, resp. the Femural Reference Point Extension Rod. Adjust the OrthoLength sliders and the Links on the Reference Points to obtain perfect geometric fit between the keel like Measuring Points and the Reference Points. This is particularly important as this first Reference Measurement serves at the measurements to which all subsequent measurements are compared.

Next, The values for offset and leg length, represented as the values of the three OrthoLength sliders, should be written down for later reference. If, based on preoperative planning, adjustments of Leg Length and/or Offset are planned the horizontal- and vertical sliders are adjusted accordingly and locked again (The Adjusted Reference Measurement). Make a written note of the values of the three sliders in the position of the Adjusted Reference Measurement.

Before proceeding, remove the Pelvic Reference Point from the Pelvic Nail, and remove the Femural Reference Point and the Extension Rod from the Trochanter Clamp.

Proceed according to normal operating technique.

Divide the femoral neck, prepare bone beds and perform a trial reduction.

### Checking Leg Length and Offset

Reduce the prosthetic joint by bringing the patient's hip and knee back into the same position as when the Reference Measurement with OrthoLength™ was taken. Use OrthoLength™ mounted on the Pelvic Reference Point to ascertain that correct position of the patient is obtained. If difficulty is experienced obtaining good axial alignment between Measuring Points and Reference Points, the position of the patient and/or the patient's leg should be adjusted to achieve good fit, thus ascertaining that the patient is back in the same position as during the reference measurement.

When the Pelvic Measuring Point is mounted and locked on the Pelvic Reference point, too much offset will be visualized as a parallel gap between the Femural Measuring- and Reference points. Too small offset will be visualized as a situation where the value of the distal vertical glider must be reduced to obtain perfect fit.

Compare the actual values for Leg Length and Offset with the values of the Adjusted Reference Measurement.

As required, adjust component position or choice of prosthesis components to obtain the planned values for leg length and offset.

Minor discrepancies in Leg Length may be read of the top of the Femur Reference Point, which is indexed.

After interim measurements, always bring the sliders back to the values of the Adjusted Reference Measurement.

When satisfied with the measurements, temporarily remove both Reference Points. Also always remove Reference Points when dislocating and reducing the joint.

Proceed according to normal operating technique.

Insert prosthesis components.

### Final Check of Leg Length and Offset

When the prosthesis is in place, hip and knee joint are extended to bring the leg back into the same position as with the Reference Measurement, it is recommended that Reference Points and OrthoLength™ are mounted for a final check of for Leg Length and Offset. Thus allowing the surgeon a final opportunity to compensate errors by choosing a prosthesis head with longer/shorter neck length.

### Adverse Effects

Performing the Reference Measurement and correctly calculating the Adjusted Reference Measurement is of utmost importance. It is recommended to WRITE DOWN THE VALUES FOR SUBSEQUENT REFERENCE

### Sterility

These devices are preferably provided sterile by gamma irradiation indicated by the “Sterile R” symbol on the exterior of the box.

OrthoLength™ is the name of the OrthoMeter™ system for preoperative surveillance of Leg Length and Offset during Total Hip Replacements. OrthoLength™ comprises a toolbox of surgical instruments and a disposable kit.

The OrthoLength™ Instrument set is supplied as high quality instruments manufactured in Stainless Steel and Titanium. Careful attention must be paid to the washing and sterilization procedures described in the separate manual: “Washing, Assembling and Sterilzation”.

### The OrthoLength™ Toolbox

The OrthoLength™ Toolbox—is a set of high quality specialised instruments used during THR-surgery.

For convenience, the instruments are described with their part number, names and relevant use.

Upon ordering and receipt of goods, please check carefully to make sure that the toolbox contents are in accordance with the specifications.

Caution: Components must be sterilized according to the procedure described in “Washing, Assembling and Sterilization”. Proper sterility is the responsibility of the user.

### Exemplary Content of a OrthoLength™ Toolbox

### Part no.

### Pelvis Adapter

### Measuring Point×2

### Pelvis Guide×2

### Femur Fixture

### The OrthoLength™ Disposable Kit

The Disposable Kit (cat. no. 201.001) consisting of:
1 OrthoLength plastic measure ring device
1 Nail for supra-acetabular mounting

The elements and components of an embodiment of the invention may be physically, functionally and logically implemented in any suitable way. Indeed, the functionality may be implemented in a single unit, in a plurality of units or as part of other functional units. As such, the invention may be implemented in a single unit, or may be physically and functionally distributed between different units.

Although the present invention has been described above with reference to a specific embodiments, it is not intended to be limited to the specific form set forth herein. Rather, the invention is limited only by the accompanying claims and, other embodiments than the specific above are equally possible within the scope of these appended claims, e.g. different arm shapes than those described above.

In the claims, the term “comprises/comprising” does not exclude the presence of other elements or steps. Furthermore, although individually listed, a plurality of means, elements or method steps may be implemented by e.g. a single unit or processor.

Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A system for ensuring correct insertion and spatial orientation of a prosthesis cup and/or a prosthesis stem of an artificial hip joint, the system comprising: a) a tool for controlling the mutual positioning of the main components in a hip prosthesis; b) a measuring device, for measuring the distance between two supports connected to the patient's pelvis and leg, comprising; an elongated main body; a first arm and a second arm, wherein said first and second arm is connected to said elongated main body and at least one of said first and second arm is displaceable along said main body along a first plane, and at least one of said first and second arm is repositionable with regard to said main body along a second plane substantially orthogonal to said first plane, wherein the relation between said first and second plane anteriorly or posteriorly may be shifted along said second plane of the measuring device; and connection members arranged at one of the ends of said first and second arm, where said connection members are adapted to interact with receptors at said supports.

2. The system according to claim 1, further comprising c) a detachable positioning tool designed to be connected to a handle part connected to the tool according to item a) or to extensions of the prosthesis components, and to the two supports connected to the patients' pelvis and leg.

3. The system according to claim 1, wherein said connection members are arranged on a sliding track in the end of said first and/or second arm.

4. The system according to claim 1, wherein said receptors are arranged on a sliding track on said supports.

5. The system according to claim 3, wherein said sliding track is curved.

6. The system according to claim 3, wherein said connection members and/or receptors are fixable on said sliding track by a fixation means.

7. The system according to claim 6, wherein said fixation means is a turning knob.

8. A measuring device for measuring the distance between two supports for use during surgical procedures, where said supports are connected to bones in the patient's body, wherein the measuring device comprises an elongated main body; a first arm and a second arm, wherein said first and second arm is connected to said elongated main body and at least one of said first and second arm is displaceable along said main body along a first plane, and at least one of said first and second arm is repositionable with regard to said main body along a second plane substantially orthogonal to said first plane, wherein the relation between said first and second plane anteriorly or posteriorly may be shifted along said second plane of the measuring device; connection members arranged at one of the ends of said first and second arm, where said connection members are adapted to interact with receptors at the supports.

9. The measuring device according to claim 8, wherein at least one of said first and second arm is rotatable in said first plane.

10. The measuring device according to claim 8, wherein said first and/or second arm is positioned on an adjustable member.

11. The measuring device according to claim 8, wherein said first arm is displaceably connected to the main body in a direction substantially perpendicular to the longitudinal axis of said main body.

12. The measuring device according to claim 11, wherein said first arm is releasably lockable and displaceable/pivotable/rotatable around an axis perpendicular to said first plane of said main body.

13. The measuring device according to claim 10, wherein said second arm is displaceably connected to the adjustable member in a direction substantially perpendicular to said first plane of said main body.

14. Measuring device according to claim 10, wherein the adjustable member comprises means to lock the adjustable member to a wanted position along the main body.

15. The measuring device according to claim 8, wherein said second arm is releasably lockable and displaceable/pivotable/rotatable around an axis perpendicular to said first plane of said main body.

16. Measuring device according to claim 10, wherein the adjustable member is adapted to releasably lockable receive and interact with a locking member to lock the adjustable member to the main body.

17. Measuring device according to claim 8, additionally comprising means to lock said first arm in a wanted position.

18. Measuring device according to claim 8, additionally comprising means to lock said second arm in a wanted position.

19. The measuring device of claim 1, wherein substantially parallel bores are made in the main body and/or the adjustable body, such that a drilling support is formed.

20. The measuring device of claim 1, wherein the adjustment means comprises locking means.

21. The measuring device of claim 1, wherein the adjustment means comprises means for reading out the orthogonal anterior or posterior shift.

22. A tool for measuring distances between components in a surgical method, the tool comprising:
an elongate main body;
two attachment members projecting from the main body, at least one of which is slideably movable along the length of the main body;
wherein the attachment members are connected to the main body so as not to be rotatable about the axis of the main body, and the attachment members can each be removably attached to a respective component for measuring the distance between the components.
